SEQUENCE LISTING (10 sequences)

SEQ ID NO: 1 - DNA, 20 bp, Artificial Sequence
  primer
aggcacaacaggctgctctg

SEQ ID NO: 2 - DNA, 20 bp, Artificial Sequence
  primer
tggaccagacatcaccaagc

SEQ ID NO: 3 - DNA, 20 bp, Artificial Sequence
  primer
agcgccttcggtccagttgc

SEQ ID NO: 4 - DNA, 20 bp, Artificial Sequence
  primer
actcatctgcacagctctgg

SEQ ID NO: 5 - DNA, 21 bp, Artificial Sequence
  primer
cgccctgttcgctctgggtat

SEQ ID NO: 6 - DNA, 21 bp, Artificial Sequence
  primer
aggaggtccgcatgctcacag

SEQ ID NO: 7 - DNA, 20 bp, Artificial Sequence
  primer
atgctcttcagttcgtgtgt

SEQ ID NO: 8 - DNA, 20 bp, Artificial Sequence
  primer
agctgacttggcaggcttgt

SEQ ID NO: 9 - DNA, 21 bp, Artificial Sequence
  primer
aagccgcatacgcacccagag

SEQ ID NO: 10 - DNA, 21 bp, Artificial Sequence
  primer
tggggtaccgctgctgggagg

(19) United States
(12) Patent Application Publication
(10) Pub. No.: US 2007/0087064 A1
(43) Pub. Date: Apr. 19, 2007

(54) Composition for preventing and treating climacteric symptoms comprising the extract of sophorae fructus

(75) Inventors: Suk-Hyung Kwon, Seoul (KR); Bo-Sik Hwang, Gyeonggi-do (KR); Kuk-Hwan Kim, Chungcheongbuk-do (KR); Seung-Hwan Lee, Seoul (KR); Eun-Ju Yoon, Daejeon (KR); Seung-Hee Lee, Chungcheongbuk-do (KR); Min-Won Lee, Seoul (KR); Do-Ik Lee, Seoul (KR); Yung-Wook Choi, Seoul (KR); Seong-Soo Joo, Gyeonggi-do (KR); Tae-Joon Won, Seoul (KR); Hee-Chul Kang, Seoul (KR)

Correspondence Address: BUCHANAN, INGERSOLL & ROONEY PC, POST OFFICE BOX 1404, ALEXANDRIA, VA, 22313-1404, US

(73) Assignee: DREXGENEBIOTECH CO., LTD., Chungcheongbuk-do (KR)

(21) Appl. No.: 10/580,469

(22) PCT Filed: Feb. 9, 2004
(86) PCT No.: PCT/KR04/00251

§ 371 Date: May 25, 2006

(30) Foreign Application Priority Data
Nov. 26, 2003 (KR) 10-2003-0084329

(51) Int. Cl. A61K 36/489

(52) U.S. Cl. 424/757; 424/777

(57) ABSTRACT

The present invention relates to a composition for preventing and treating climacteric symptoms comprising the extract of Sophorae Fructus as an effective ingredient. Precisely, the present invention relates to the extract of Sophorae Fructus having an effect of preventing and treating climacteric symptoms, a composition comprising the extract and a method for preventing and treating climacteric symptoms by administering the composition. The extract of Sophorae Fructus of the present invention has activities of promoting the osteoblast proliferation, inhibiting the secretion of bone-absorptive cytokines, promoting the secretion of growth factors involved in bone-reformation, stimulating the generation of nitric oxide in osteoblasts and inhibiting the osteoclast differentiation. In addition, the extract also reduces the concentration of a bone absorption index and inhibits the decrease of calcium content and bone density. Therefore, the extract of Sophorae Fructus of the present invention can be effectively used for the prevention and the treatment of climacteric symptoms including osteoporosis.

The application claims the priority of Korean Patent Application No. 10-2003-0084329 filed on Nov. 26, 2003.

### FIELD OF THE INVENTION

The present invention relates to a composition and method for preventing and treating climacteric symptoms including osteoporosis.

### BACKGROUND OF THE INVENTION

Climacteric symptoms are caused by the decrease of secretion of male or female hormones. Especially, in the case of women, estrogen is less secreted as an ovary gets old, resulting in climactic symptoms for about 2˜10 years before and after menopause. High fever, sweat, insomnia, depression, urinary incontinence, pain, osteoporosis, myocardial infraction, cerebral apoplexy and hypertension are the representative climacteric symptoms.

Among those symptoms, osteoporosis is the most typical one, which is caused by the decrease of total bone mass induced as osteoclasts exceed osteoblasts in their activities. Once osteoporosis is developed, the width of cortical bone becomes narrower, the cavity of bone marrow is expanded and bone column of reticular tissue becomes lower, resulting in porosity in bones. As osteoporosis gets serious, physical strength of bones is further declined, causing lumbago, arthralgia and bone breaking even by a slight impact.

Until now, the methods to prevent and treat climacteric symptoms such as hormone replacement therapy, non-steroid medicines and medicinal therapy for osteoporosis, etc. have been developed. The most effective method of them seems to be hormone replacement therapy. However, long-term administration of a hormone carries side effects such as headache, gaining weight, possibility of tumorigenesis, etc. Therefore, a safer and more effective treatment agent or method is required.

Recently, studies have been actively conducted to develop a novel substance having an excellent pharmaceutical effect with fewer side effects during long-term administration, so as to substitute estrogen. One of the attractive candidates for substituting estrogen is phytoestrogen, which is included in soybeans, etc. The phytoestrogen has a similar structure to human estrogen, so that it has influence on diseases involved in hormone or anti-hormone activities in vivo. So, the possibility of using phytoestrogen as a food supplementary agent to take the place of hormone replacement therapy has been examined. The representative phytoestrogens, known so far, are isoflavone compounds such as daidzein, genistein, formononetin, biochanin A, etc., coumestan compounds like coumestrol, etc., lignan compounds such as enterolactone, etc., and phenol compounds like enterodiol, etc.

Korea patent No. 348148 discloses the extract of Pueraria root having huge amount of phytoestrogen and preventing and treating effects for osteoporosis. In addition, the extract of Pueraria root has also been reported to have enough amount of daidzein, a kind of phytoestrogen, to have influence on the prevention and treatment of osteoporosis (Kim C. S. et al., Korean J. Food Sci. Technol., 34(4), 710˜718, 2002). Soybean powder has been reported to have an effect of improving osteoporosis, too (Yang S. B. et al., Korean Journal of Bone Metabolism, 6(1), 11˜17, 1999).

Sophorae Fructus is a fruit of a Sophora japonica Linne. The Sophora japonica Linne, a deciduous arbor, belongs to a pea family (Leguminosae), and largely inhabits Korea, Japan and China The contents are varied from the parts of the tree and have different medical actions content by content.

Sophorae Flos, a flower of a Sophora japonica Linne, is known to have such medicinal actions as anti-inflammation, anti-ulcer, declining of blood pressure, and preventing and treating effects of arteriosclerosis (Kim C. M. et al., Dictionary of Traditional Chinese Medicine, Vol. 1, Jungdam Publishing, 496˜509, 1998).

Sophorae resina, a resin of a Sophora japonica Linne, has been used for treatment of tetanus. All the leaves, branches, bark and root bark of the Sophora japonica Linne have an antimicrobial activity (Yook C. S. et al., K. H. Pharma. Sci., 17, 75˜87, 1989).

Sophorae Fructus, a fruit of Sophora japonica Linne, has a blood sugar increasing activity and an antimicrobial activity, and thus has been used for treatment of hemorrhoids, uterine hemorrhage, hematuria, hematemesis, hemoptysis and anal prolapse (Kim C. M. et al., Dictionary of Traditional Chinese Medicine, Vol. 1, Jungdam Publishing, 496˜509, 1998).

### SUMMARY OF THE INVENTION

The present inventors have endeavored to find a novel substance available for prevention and treatment of climacteric symptoms without side effects, and have completed the invention by confirming that an extract of Sohporae Fructus, a fruit of Sophora japonica Linne, has an excellent activity of preventing and treating climacteric symptoms.

Thus, it is an object of the present invention to provide a pharmaceutical composition for preventing or treating climacteric symptoms comprising the extract of Sophorae Fructus as an effective ingredient.

It is another object of the present invention to provide a food composition for preventing or improving climacteric symptoms comprising the extract of Sophorae Fructus as an effective ingredient.

It is another object of the present invention to provide a method for preventing or treating climacteric symptoms, which comprises administering a pharmaceutical composition comprising the extract of Sophorae Fructus to a subject.

It is another object of the present invention to provide a method for preventing weight gaining, which comprises administering a pharmaceutical composition comprising the extract of Sophorae Fructus to a subject.

It is another object of the present invention to provide a use of the extract of Sophorae Fructus for the preparation of a medicament for preventing or the treating climacteric symptoms.

### BRIEF DESCRIPTION OF THE DRAWINGS

FIG. 1 shows the effect of an extract of Sophorae Fructus of the present invention on the osteoblst proliferation, confirmed by MTT method (R-G: a group treated with an extract of Sophorae Fructus of Example 1, R-A: a group treated with an enzyme extract of Sophorae Fructus of Example 2, R-P: a group treated with a food composition comprising an extract of Sophorae Fructus of Example 3, S-S: a group treated with soybean ex-powder, E: a group treated with 17-beta estradiol, control: a group treated with cell culture medium, LPS: a group treated with lipopolysaccharide).

FIG. 2A shows the IL-1 beta secretion inhibitory effect of an extract of Sophorae Fructus of the present invention, confirmed by ELISA (R-G: a group treated with an extract of Sophorae Fructus of Example 1, R-A: a group treated with an enzyme extract of Sophorae Fructus of Example 2, R-P: a group treated with a food composition comprising an extract of Sophorae Fructus of Example 3, S-S: a group treated with soybean ex-powder, E: a group treated with 17-beta estradiol, control: a group treated with cell culture medium).

FIG. 2B shows the IL-6 secretion inhibitory effect of an extract of Sophorae Fructus of the present invention, confirmed by ELISA (R-G: a group treated with an extract of Sophorae Fructus of Example 1, R-A: a group treated with an enzyme extract of Sophorae Fructus of Example 2, R-P: a group treated with a food composition comprising an extract of Sophorae Fructus of Example 3, S-S: a group treated with soybean ex-powder, E: a group treated with 17-beta estradiol, control: a group treated with cell culture medium).

FIG. 3 shows the expressions of IL-1 beta and IL-6 inhibited by an extract of Sophorae Fructus of the present invention, confirmed by RT-PCR (R-G: a group treated with an extract of Sophorae Fructus of Example 1, R-A: a group treated with an enzyme extract of Sophorae Fructus of Example 2, R-P: a group treated with a food composition comprising an extract of Sophorae Fructus of Example 3, S-S: a group treated with soybean ex-powder, E: a group treated with 17-beta estradiol).

FIG. 4A presents the result of ELISA showing the IGF-1 secretion promoting effect of an extract of Sophorae Fructus of the present invention (R-G: a group treated with an extract of Sophorae Fructus of Example 1, R-A: a group treated with an enzyme extract of Sophorae Fructus of Example 2, R-P: a group treated with a food composition comprising an extract of Sophorae Fructus of Example 3, S-S: a group treated with soybean ex-powder, E: a group treated with 17-beta estradiol, control: a group treated with cell culture medium).

FIG. 4B presents the result of ELISA showing the TGF-β secretion promoting effect of an extract of Sophorae Fructus of the present invention (R-G: a group treated with an extract of Sophorae Fructus of Example 1, R-A: a group treated with an enzyme extract of Sophorae Fructus of Example 2, R-P: a group treated with a food composition comprising an extract of Sophorae Fructus of Example 3, S-S: a group treated with soybean ex-powder, E: a group treated with 17-beta estradiol, control: a group treated with cell culture medium).

FIG. 5 presents the result of RT-PCR reflecting the expression of IGF-1 and TGF-β induced by an extract of Sophorae Fructus of the present invention (R-G: a group treated with an extract of Sophorae Fructus of Example 1, R-A: a group treated with an enzyme extract of Sophorae Fructus of Example 2, R-P: a group treated with a food composition comprising an extract of Sophorae Fructus of Example 3, S-S: a group treated with soybean ex-powder, E: a group treated with 17-beta estradiol).

FIG. 6 presents the result of ELISA showing the nitric oxide (NO) generation promoting effect of an extract of Sophorae Fructus of the present invention (R-G: a group treated with an extract of Sophorae Fructus of Example 1, R-A: a group treated with an enzyme extract of Sophorae Fructus of Example 2, R-P: a group treated with a food composition comprising an extract of Sophorae Fructus of Example 3, S-S: a group treated with soybean ex-powder, E: a group treated with 17-beta estradiol, control: a group treated with cell culture medium).

FIG. 7 presents the result of RT-PCR showing the level of expression of endothelial nitric oxide synthase (ecNOS) induced by an extract of Sophorae Fructus of the present invention. GAPDH was used for loading control (R-G: a group treated with an extract of Sophorae Fructus of Example 1, R-A: a group treated with an enzyme extract of Sophorae Fructus of Example 2, R-P: a group treated with a food composition comprising an extract of Sophorae Fructus of Example 3, S-S: a group treated with soybean ex-powder, E: a group treated with 17-beta estradiol, *:R-P, E vs S-S, statistically significant when p<0.05).

FIG. 8 presents the number of positive osteoclasts observed under an optical microscope after staining with TRAP to measure a osteoclast differentiation inhibitory activity of an extract of Sophorae Fructus of the present invention (R-G: a group treated with an extract of Sophorae Fructus of Example 1, R-A: a group treated with an enzyme extract of Sophorae Fructus of Example 2, R-P: a group treated with a food composition comprising an extract of Sophorae Fructus of Example 3, E: a group treated with 17-beta estradiol, S-S: a group treated with soybean ex-powder).

FIG. 9 presents a osteoclast differentiation inhibitory activity of an extract of Sophorae Fructus of the present invention, which was analyzed by measuring optical density after staining with TRAP (Control: a group treated with cell culture medium, R-G: a group treated with an extract of Sophorae Fructus of Example 1, R-A: a group treated with an enzyme extract of Sophorae Fructus of Example 2, R-P: a group treated with. a food composition comprising an extract of Sophorae Fructus of Example 3, S-S: a group treated with soybean ex-powder, E: a group treated with 17-beta estradiol).

FIG. 10 is a graph showing the weight changes in ovari-ectomized rats administered with an extract of Sophorae Fructus of the present invention (E: a group administered with 17-beta estradiol, R-G: a group administered with an extract of Sophorae Fructus of Example 1, R-A: a group administered with an enzyme extract of Sophorae Fructus of Example 2, S-S: a group administered with soybean ex-powder).

FIG. 11 is a calibration curve showing the relations between Dpd (Deoxypyridinoline) concentration and optical density, by which Dpd concentration in a blood plasma of ovari-ectomized rats administered with an extract of Sophorae Fructus of the present invention, could be measured (Y=−0.1128X+1.6102, R=0.9902).

FIG. 12 is a graph showing the changes of Dpd concentration in a blood plasma of ovari-ectomized rats administered with an extract of Sophorae Fructus of the present invention (E: a group administered with 17-beta estradiol, R-A: a group administered with an enzyme extract of Sophorae Fructus of Example 2, R-G: a group administered with an extract of Sophorae Fructus of Example 1, R-P: a group administered with a food composition comprising an extract of Sophorae Fructus of Example 3, S-S: a group treated with soybean ex-powder).

FIG. 13 is a graph showing the difference between before and after experiments in Dpd concentration in a blood plasma of ovari-ectomized rats administered with an extract of Sophorae Fructus of the present invention (Control: a group administered with water, E: a group administered with 17-beta estradiol, R-A: a group administered with an enzyme extract of Sophorae Fructus of Example 2, R-G: a group administered with an extract of Sophorae Fructus of Example 1, R-P: a group administered with a food composition comprising an extract of Sophorae Fructus of Example 3, S-S: a group administered with soybean ex-powder).

FIG. 14 is a graph showing the comparison of Dpd inhibitory activity of an extract of Sophorae Fructus of the present invention (E: a group administered with 17-beta estradiol, R-G: a group administered with an extract of Sophorae Fructus of Example 1, S-S: a group administered with soybean ex-powder, R-P: a group administered with a food composition comprising an extract of Sophorae Fructus of Example 3, R-A: a group administered with an enzyme extract of Sophorae Fructus of Example 2, *: not statistically significant when p<0.05, **: statistically significant when p<0.05).

FIG. 15 is a graph showing the changes of calcium concentration in a blood plasma of ovari-ectomized rats administered with an extract of Sophorae Fructus of the present invention (E: a group administered with estradiol, R-A: a group administered with an enzyme extract of Sophorae Fructus of Example 2, R-G: a group administered with an extract of Sophorae Fructus of Example 1, R-P: a group administered with a food composition comprising an extract of Sophorae Fructus of Example 3, S-S: a group administered with soybean ex-powder).

FIG. 16A is a microphotograph showing the tibia of ovari-ectomized rats administered with an extract of Sophorae Fructus of the present invention (Magnification: ×16, A: a normal group (non-ovari-ectomized group), B: control 1 (sham-operated group), C: control 2 (ovari-ectomized group), D: a group administered with 17-β estradiol, E: a group administered with an extract of Sophorae Fructus of Example 1, F: a group administered with an enzyme extract of Sophorae Fructus of Example 2, G: a group administered with a food composition comprising an extract of Sophorae Fructus of Example 3, H: a group administered with soybean ex-powder).

FIG. 16B presents the area of trabecular bone of the tibia of ovari-ectomized rats administered with an extract of Sophorae Fructus of the present invention (E: a group administered with 17-beta estradiol, R-A: a group administered with an enzyme extract of Sophorae Fructus of Example 2, R-G: a group administered with an extract of Sophorae Fructus of Example 1, R-P: a group administered with a food composition comprising an extract of Sophorae Fructus of Example 3, S-S: a group administered with soybean ex-powder, *: statistically significant when p<0.05).

FIG. 17A is a microphotograph showing the lumbar of ovari-ectomized rats administered with an extract of Sophorae Fructus of the present invention (Magnification: ×16, A: a normal group (non-ovari-ectomized group), B: control 1 (sham-operated group), C: control 2 (ovari-ectomized group), D: a group administered with 17-β estradiol, E: a group administered with an extract of Sophorae Fructus of Example 1, F: a group administered with an enzyme extract of Sophorae Fructus of Example 2, G: a group administered with a food composition comprising an extract of Sophorae Fructus of Example 3, H: a group administered with soybean ex-powder).

FIG. 17B presents the area of trabecular bone of the lumbar of ovari-ectomized rats administered with an extract of Sophorae Fructus of the present invention (E: a group administered with 17-beta estradiol, R-A: a group administered with an enzyme extract of Sophorae Fructus of Example 2, R-G: a group administered with an extract of Sophorae Fructus of Example 1, R-P: a group administered with a food composition comprising an extract of Sophorae Fructus of Example 3, S-S: a group administered with soybean ex-powder, *: statistically significant when p<0.05).

### DETAILED DESCRIPTION OF THE INVENTION

In order to achieve the above objects of the invention, the present invention provides a pharmaceutical composition for preventing or treating climacteric symptoms comprising the extract of Sophorae Fructus as an effective ingredient.

The present invention also provides a food composition for preventing or improving climacteric symptoms comprising the extract of Sophorae Fructus as an effective ingredient.

The present invention also provides a method of preventing or treating climacteric symptoms, which comprises administering a pharmaceutical composition comprising the extract of Sophorae Fructus to a subject.

The present invention also provides a method of preventing weight gaining, which comprises administering a pharmaceutical composition comprising the extract of Sophorae Fructus to a subject.

The present invention furter provides a use of the extract of Sophorae Fructus for the preparation of a medicament for preventing or treating climacteric symptoms.

The present invention will be described in detail.

“Sophorae Fructus” of the present invention refers to a friuit of Sophora japonica Linne, a deciduous arbor belonging to a pea family (Leguminosae). More particularly, it means a mature fruit of Sophora japonica Linne.

It is preferable for the present invention that Sophorae Fructus, as a mature fruit of Sohpora japonica Linne, ought to have its unique color and flavor without other taste and smell. The peel of the fruit has to be khaki brown or brown, and a seed had better be black or black brown.

It is preferable to prepare an extract of Sophorae Fructus of the present invention by hydrothermal extraction, but not always limited thereto. The ratio of Sophorae Fructus to water for hydrothermal extraction is not specially limited, but for 1 g of Sophorae Fructus, water can be used by 3 to 20 times (based on weight) preferably, 5 to 10 times.

The temperature for extraction is preferably room temperature under atmospheric pressure. The extraction time varies depending on extraction temperature, but preferably ranges from 1 to 6 hours, more preferably 2 to 4 hours. Also, extraction efficiency may further enhanced by stirring with a shaker during extraction.

Sophorae Fructus can be used either right after being rinsed after cropping or after being dried. Sophorae Fructus can be dried either in the sun, in the shade, by hot air or naturally. In addition, Sophorae Fructus or its dried body can be crushed into powder to enhance the efficiency of the extraction.

Preferably, dried Sophorae Fructus can be pulverized in 20˜40 mesh size, and drinking water is added to the Sophorae Fructus powder, wherein the ratio of Sophorae Fructus powder to water is 1 to 3˜20, preferably 1 to 5˜10. Then, hydrothermal extraction is carried out for 1 to 3 hours at 100˜130° C., preferably 120˜125° C. The Sophorae Fructus extract can be prepared by centrifuging the hydrothermal extract and removing the precipitation to obtain supernatant.

An enzyme extract of Sophorae Fructus is also obtained by treating the hydrothermal extract of Sophorae Fructus of the invention with an enzyme. Precisely, the hydrothermal extract prepared by the above method is treated with an enzyme by 0.01˜1 %(v/v), followed by a reaction for 4˜24 hours. After concentration, the reaction solution is freeze-dried, resulting in an enzyme extract. At this time, one of α-amylase, β-amylase and pectinase can be used as an enzyme.

The extract of Sophorae Fructus of the present invention has an effect of preventing and treating climacteric symptoms. The “climacteric symptoms” as used herein refers to diseases that can be caused by the lack of a hormone, and especially for women, they are caused by the deficiency in estrogen, which results from the blockage of functions of ovary. The representative climacteric symptoms are classified into metabolic bone diseases such as osteoporosis, lumbago, rheumatoid arthritis, degenerative arthritis, rickets, osteomalacia and Paget's disease of bone, cardiovascular diseases such as angina pectoris and arteriosclerosis, and degenerative neurological diseases such as Parkinson's disease. Particularly, the metabolic bone disease is developed by the break of balance between osteoclasts and osteoblasts, and osteoporosis is the most representative one. The extract of Sophorae Fructus of the present invention has an excellent effect of preventing and treating osteoporosis.

The effect of preventing and treating climacteric symptoms of the extract of Sophorae Fructus of the present invention has been confirmed by in vitro and in vivo experiments.

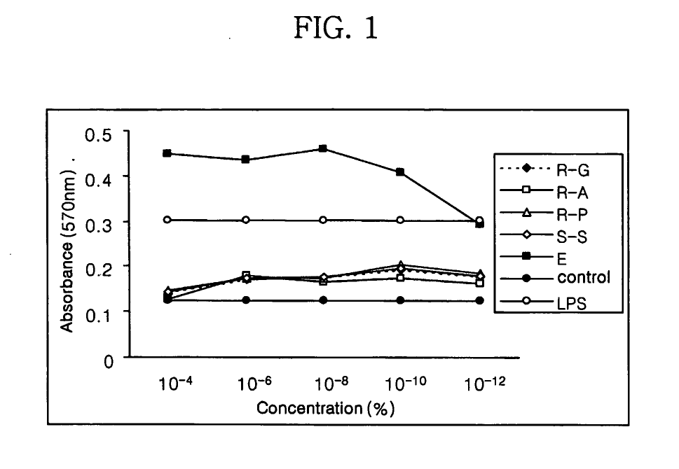

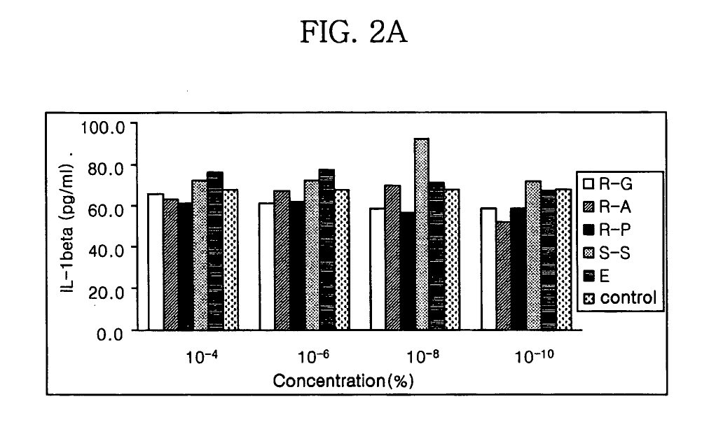

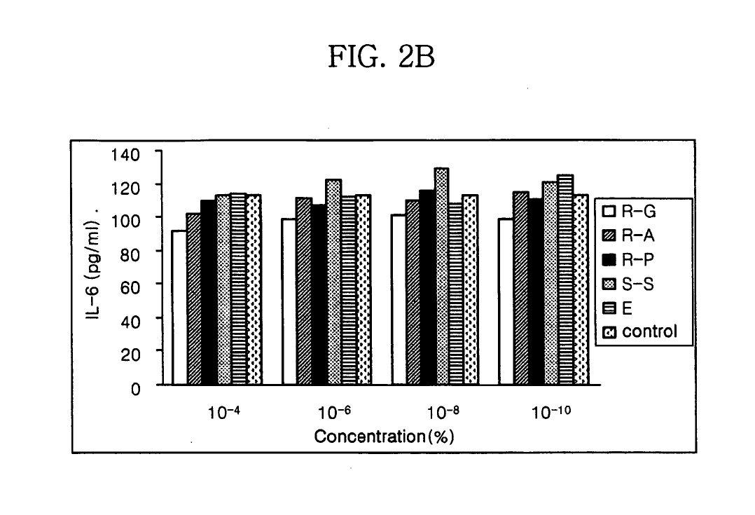

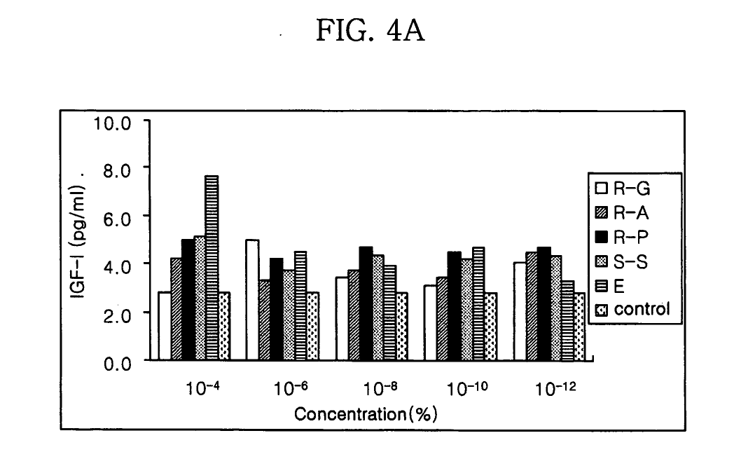

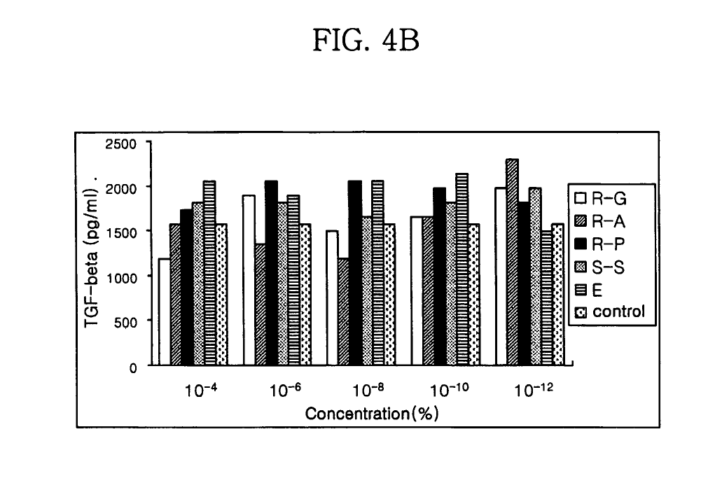

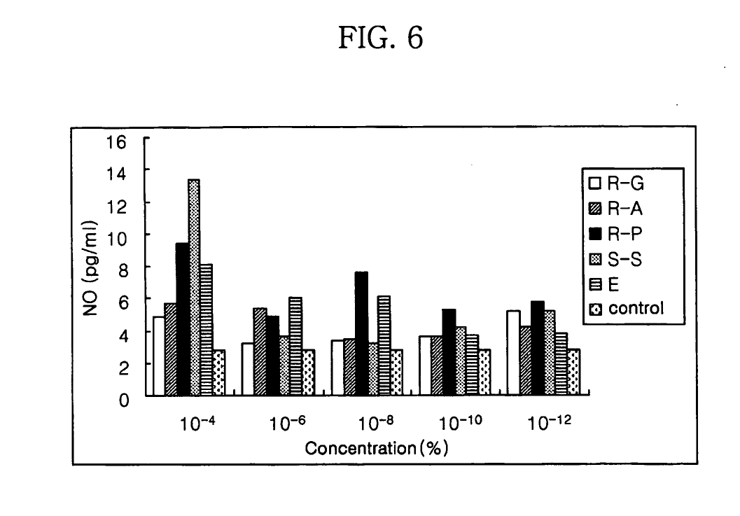

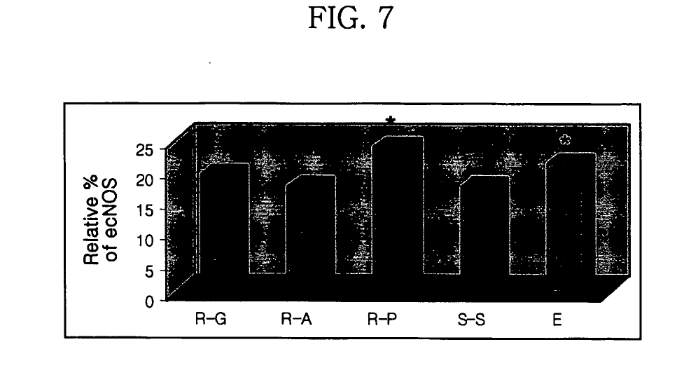

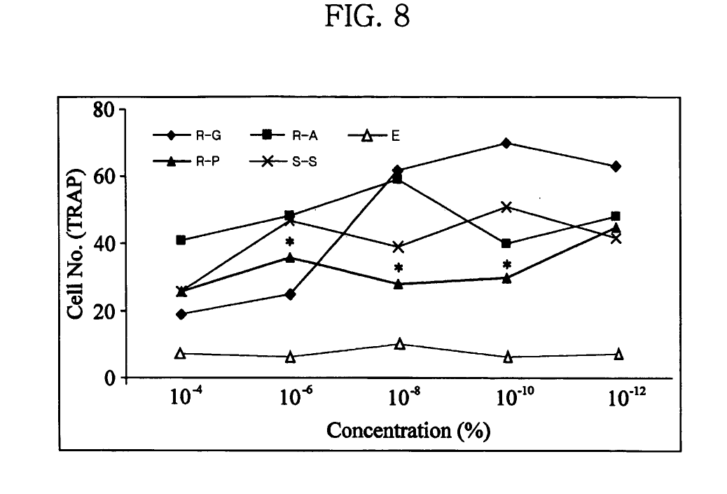

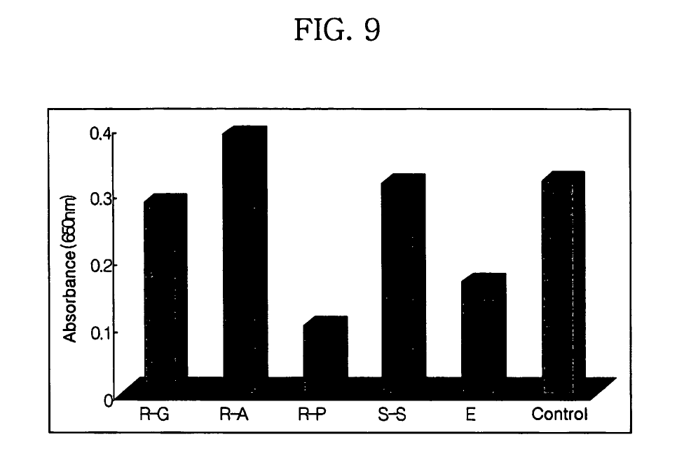

Through in vitro experiments, the extract of Sophorae Fructus of the present invention was proved to promote osteoblast proliferation (see FIG. 1) but inhibit the secretion of bone-absorptive cytokines, IL-1 beta and IL-6 (see FIG. 2A, 2B and 3). Besides, the extract of Sophorae Fructus of the present invention promoted the expression of IGF-1 and TGF-β which are growth factors involved in bone-regeneration (see FIG. 4A, 4B and 5), accelerated the generation of nitric oxide (see FIG. 6 and 7), and effectively inhibited osteoclast differentiation (see FIG. 8 and 9). Such activities were observed well even under the low concentration of the extract of Sophorae Fructus of the present invention.

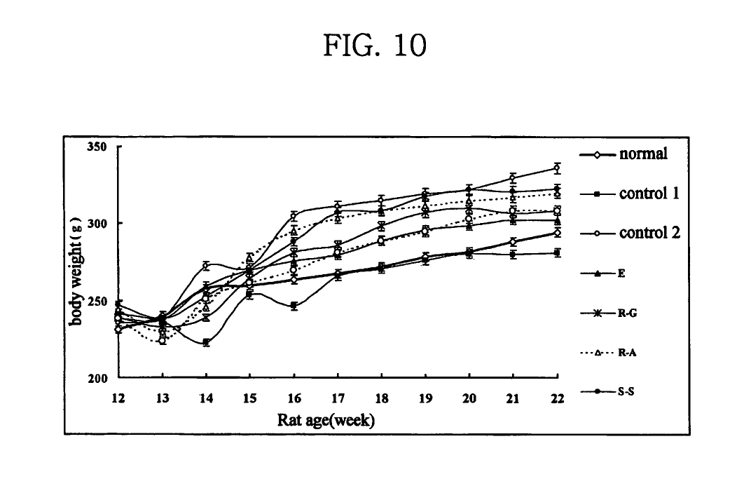

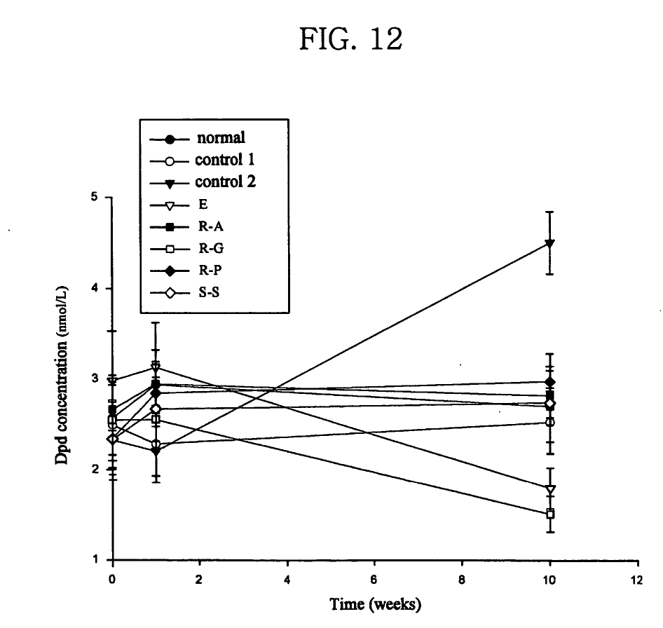

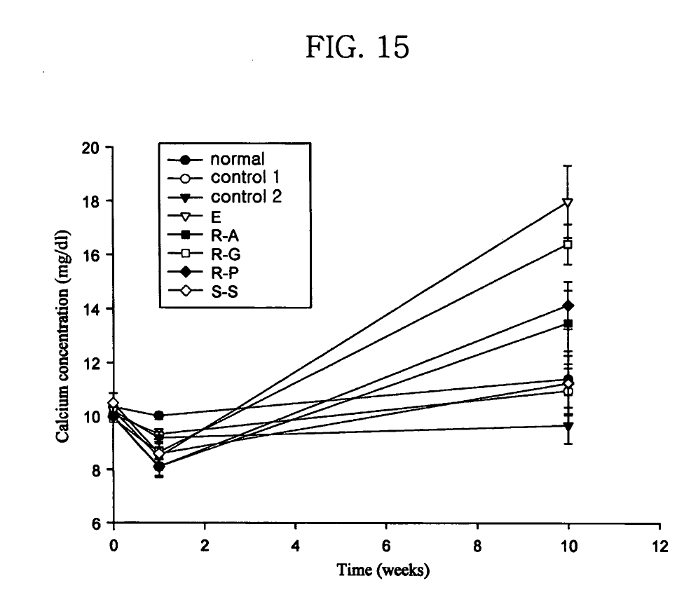

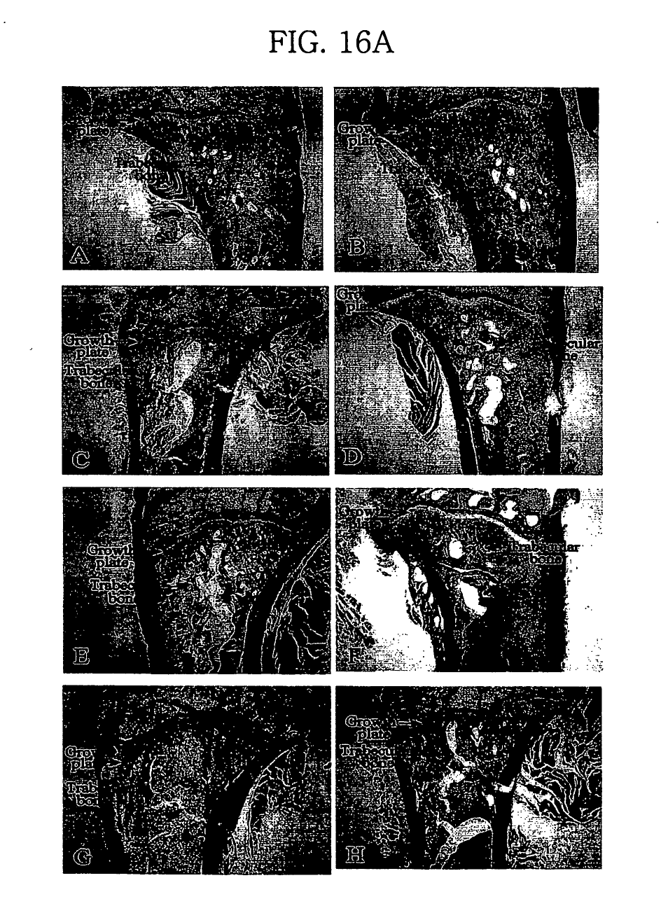

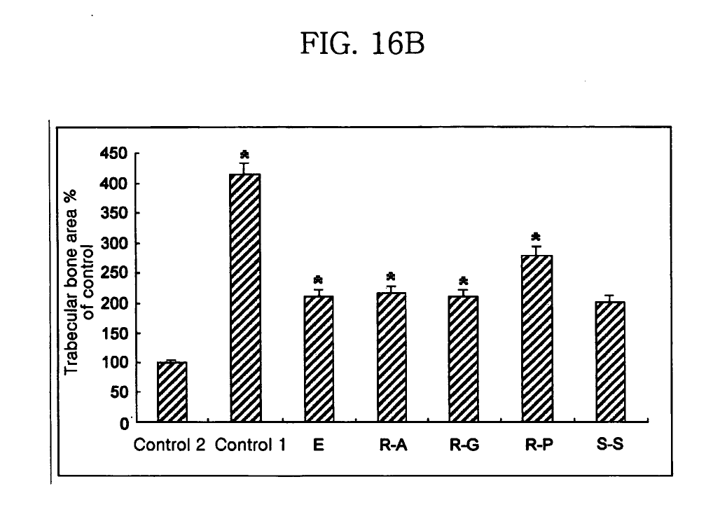

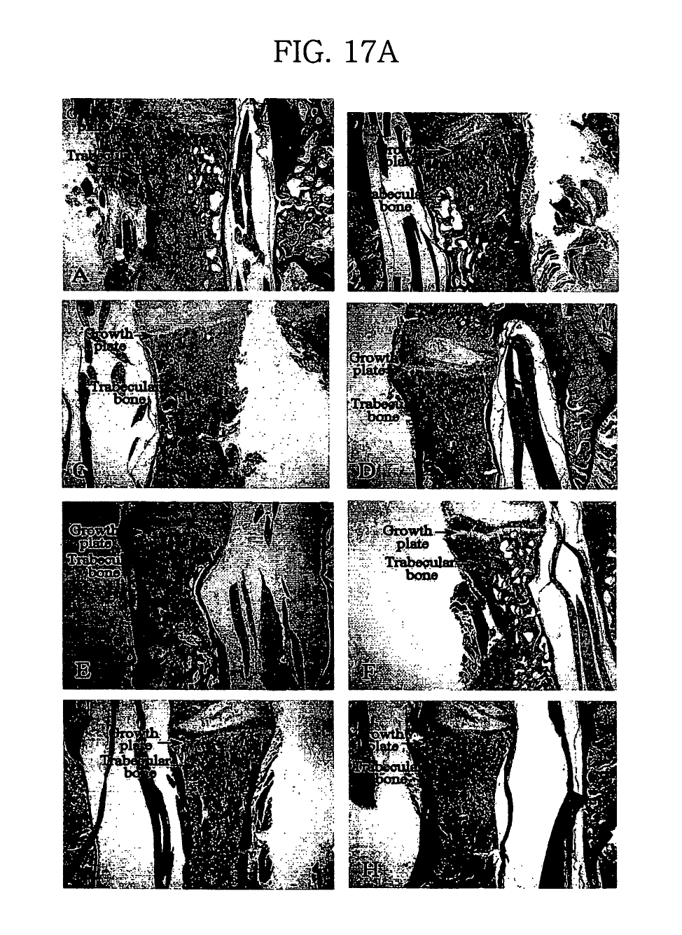

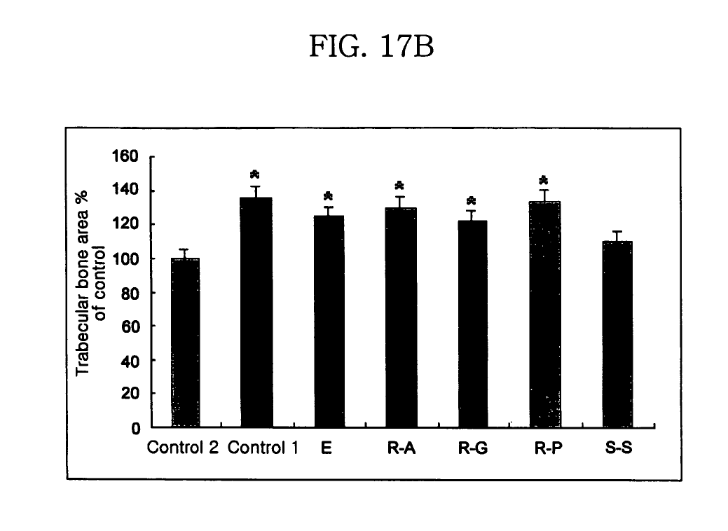

For in vivo experiments with the extract of Sophorae Fructus, ovari-ectomized rats were used. The extract of Sophorae Fructus of the present invention was administered to rats that could not secret estrogen because its ovary was removed. Then, weight changes, the level of Dpd, an index for bone replacement rate in a serum, which increases as bone matrixes are decomposed by osteoclasts, and calcium concentration varying with the activation of osteoblasts are investigated. As a result, the extract of Sophorae Fructus of the present invention worked as a substitute for estrogen and so prevented weight gaining (see FIG. 10), inhibited the increase of Dpd (see FIG. 12-FIG. 14) and increased calcium concentration in blood (see FIG. 15). In addition, the extract of Sophorae Fructus of the present invention was confirmed to inhibit the restriction of trabecular bone of the tibia and the lumbar, which can be used as an index for bone density in ovari-ectomized rats (see FIG. 16 and 17).

Thus, the present invention provides a pharmaceutical composition for preventing or treating of climacteric symptoms comprising the extract of Sophorae Fructus as an effective ingredient. The climacteric symptoms include all diseases induced by the lack of hormones, especially estrogen. For example, metabolic bone diseases such as osteoporosis, lumbago, rheumatoid arthritis, degenerative arthritis, rickets, osteomalacia and Paget's disease of bone, cardiovascular diseases such as angina pectoris. and arteriosclerosis, and degenerative neurological diseases such as Parkinson's disease, etc., are included in the symptoms. And osteoporosis is the most representative one.

The pharmaceutical composition of the present invention can include a pharmaceutically effective amount of the extract of Sophorae Fructus of the invention singly or additionally include one or more pharmaceutically acceptable carriers, binders or diluents. The term of “pharmaceutically effective amount” as used herein means the amount of an extract enough to prevent or treat the symptoms.

The pharmaceutically effective amount of the extract of Sophorae Fructus of the present invention was determined to be 1˜600 mg/day/weight kg in this invention, and more preferably 1˜100 mg/day/weight kg. Though, the effective amount can vary depending on seriousness of a disease, age, weight, body condition and sex of a patient, administration methods, and duration of treatment, etc.

The term of “pharmaceutically acceptable” as used herein means a composition that can be physiologically acceptable for humans, and does not cause side effects such as stomach trouble, allergic reactions like dizziness, etc., as being administered to humans. The carriers, binders and diluents are exemplified by lactose, dextrose, sucrose, sorbitol, manitol, xylitol, erythritol, maltitol, starch, acacia gum, alginate, gelatin, calcium phosphate, calcium silicate, cellulose, methylcellulose, polyvinylpyrolidon, water, methylhydroxybenzoate, propylhydroxybenzoate, talc, magnesium stearate and mineral oil.

The mentioned pharmaceutical composition can further include fillers, anticoagulants, lubricants, wetting agents, perfumes, emulsifying agents and antiseptics. The pharmaceutical composition of the present invention can also be formulated by the known methods in the pertinent art, to give satisfactory results after administration, for example, immediate absorption, sustaining or delayed release of an active ingredient. The composition can be formulated into the forms of powder, granule, tablet, emulsion, syrup, aerosol, soft or hard gelatin capsule, sterilized ampoule and sterilized powder.

The pharmaceutical composition of the present invention can be administered via several rouds including oral, intracutaneous, subcutaneous, intravenous or intramuscular. The effective dosage can be determined by considering administration method, age, sex, weight and seriousness of a disease of a patient, etc.

The pharmaceutical composition of the present invention can be administered along with other general compositions having an effect of preventing or treating climacteric symptoms. Natural vitamin D3, estrogen, alendronate and raloxifene are the examples of the compositions.

The extract of Sophorae Fructus of the present invention can also be added to food for prevention or treatment of climacteric symptoms. Thus, the present invention also provides a food composition comprising the extract of Sophorae Fructus of the invention as an effective ingredient. The food composition of the present invention includes all the following types of food; functional food, nutritional supplement, health food and food additives. The mentioned food composition can be produced in various forms of food by the known methods in the pertinent art.

As health food, the extract of Sophorae Fructus of the present invention can be produced in the form of tea, juice or drink, and further, granule, capsule or powder. In addition, the extract of Sophorae Fructus of the present invention can be mixed with other active ingredients generally known to have an effect of preventing and treating climacteric symptoms to produce a composition.

Functional food can be produced by adding the extract of Sophorae Fructus of the present invention to beverages (including alcoholic drinks), fruits and their processed foods (for example: canned food, bottled food, jam, marmalade, etc.), fish, meat and its processed food (for example: ham, sausage, corned beef, etc.), bread, noodles (for example: thick wheat noodle, buckwheat noodle, instant noodle, spaghetti, macaroni, etc.), fruit juice, various drinks, cookies, wheat-gluten, dairy products (for example: butter, cheese, etc.), vegetable oil, margarine, vegetable proteins, retort food, frozen food and various seasonings (for example: soybean paste, soy sauce, sauce, etc.).

As a food additive, the extract of Sophorae Fructus of the present invention can be produced in the form of powder or concentrate.

The preferable content of the extract of Sophorae Fructus of the present invention in the food composition is 30˜50 g/100 g of food.

The food composition comprising the extract of Sophorae Fructus of the present invention as an effective ingredient has an effect on osteoporosis. The extract of Sophorae Fructus of the present invention can also be produced as health food by being mixed with other active ingredients stimulating calcium absorption in vivo.

The food composition comprising the extract of Sophorae Fructus of the present invention as an effective ingredient preferably consists of the extract of 30˜50 weight % of Sophorae Fructus, 30˜50 weight % of seaweed calcium powder, 1˜10 weight % of crystalline cellulose, 0.1-2 weight % of hydrolyzed milk protein, 0.1˜2 weight % of green tea ex-powder, 0.1˜2 weight % of shark cartilage extract powder, 0.1˜2 weight % of chito-oligosaccharide, 0.1˜2 weight % of vitamin C, 0.1˜2 weight % of collagen peptide, 0.1˜2 weight % of grape seeds extract powder, 0.1˜2 weight % of enzyme mixture comprising amylase, protease, cellulase, lipase and lactase, 0.1˜0.3 weight % of vitamin D3 powder, and 0.1˜2 weight % of magnesium stearate.

Precisely, the seaweed calcium is extracted from a Rholophyta such as Porphyra tenera, Gelidium amansii and Glolopeltis tenax, and contains calcium, which is essential for bone growth, but also enough amount of magnesium, zinc, iron, fluorine, manganese, iodine and selenium. The effect of the extract of Sophorae Fructus of the present invention on prevention and treatment of osteoporosis can be enhanced by adding the said seaweed calcium powder, as a source of calcium by 30˜50 weight % to a food composition of the present invention.

Crystalline cellulose, a excipient, can be added by 1˜10 weight %.

Hydrolyzed milk protein is hydrolyzed by an enzyme or an acid to obtain an edible hydrolyzed milk protein, which includes caseinphosphopeptide (CPP) stimulating calcium absorption in vivo. So, the effect of the extract of Sophorae Fructus, included in the composition of the invention, on prevention and treatment of osteoporosis can be promoted by adding hydrolyzed milk protein by 0.1˜2 weight %. It is preferable for the said hydrolyzed milk protein to have caseinphosphopeptide (CPP) over 12%.

Green tea ex-powder and grape seeds extract powder contain a huge amount of polyphenol that prevents bone loss by inhibiting oxidation and inflammation. The effect of the extract of Sophorae Fructus, included in the composition of the present invention, on prevention and treatment of osteoporosis can be enhanced by adding green tea ex-powder and grape seeds extract powder by 0.1˜2 weight %.

Shark cartilage extract powder contains chondroitin, an essential constructing factor of cartilage. Accordingly, it is very useful for prevention of osteoporosis. So, the effect of the extract of Sophorae Fructus, included in the composition of the present invention, on prevention and improvement of osteoporosis can be enhanced by adding the shark cartilage extract powder by 0.1˜2 weight %.

Chito-oligosaccharide is a natural low-molecular polysaccharide having an enhanced coefficient of utilization in vivo, resulting from resolving chitin or chitosan obtained from a shell of Crustacea such as a crab or a shrimp, etc. Owing to excellent solubility in water, chito-oligosaccharide is absorbed well in vivo, and so can have a variety of high functional physiological activities such as immune enhancement activity, anticancer activity, antimicrobial activity, inhibiting of blood sugar increase, promoting of calcium absorption, etc. Calcium absorption can be enhanced by adding chito-oligosaccharide to the composition of the present invention by 0.1˜2 weight %. Chito-oligosaccharide having over 70% content is preferably used.

Vitamin C and vitamin D3 are known to promote calcium absorption. So, calcium absorption can be enhanced by adding vitamin C and vitamin D3 to the composition of the present invention by 0.1˜2 weight % and 0.1˜0.3 weight %, respectively.

Collagen peptide has an effect on bone formation and bone growth. So, it is also helpful to add collagen peptide to the composition of the present invention by 0.1˜2 weight %.

For an enzyme mixture of amylase, protease, cellulase, lipase and lactase, an enzyme complex labeled ‘Enerzyme-P’ might be purchased and used. The ‘Enerzyme-p’ has been used as a major raw material for uncooked food promoting digestion and adsorption, energy efficiency and metabolism. So, the composition of the present invention can be better digested and absorbed in vivo by adding the said enzyme mixture to the composition by 0.1˜2 weight %.

Magnesium stearate, a useful ingredient in joint, is a source of mucopolysaccharide, collagen and calcium. The effect of Sophorae Fructus of the present invention on preventing and improving osteoporosis can be enhanced by adding magnesium stearate to the composition of the present invention by 0.1˜2 weight %.

A food composition, prepared by mixing an extract of Sophorae Fructus of the present invention and the said calcium sources and other ingredients stimulating calcium absorption in vivo all together, has an enhanced effect on prevention and treatment of climacteric symptoms, especially osteoporosis.

The present invention also provides a method for preventing and treating climacteric symptoms, which comprises administering an effective amount of a pharmaceutical composition comprising the extract of Sophorae Fructus of the present invention to a subject.

‘A subject’ herein means mammals including humans. An effective dose’ in this invention means the amount of a composition enough to prevent or treat a disease, and the effective amount is preferably 1˜600 mg/day/weight kg, more preferably 1˜100 mg/day/weight kg. However, the effective amount can be vary depending on a disease and its seriousness, age, weight, health condition and sex of a patient, administration method, duration of treatment, etc. The administration method of the composition of the present invention is not limited specially, and general administration methods well known in the pertinent can be used.

In addition to the diseases mentioned above, climacteric symptoms include metabolic bone diseases.

A pharmaceutical composition comprising the extract of Sophorae Fructus of the present invention is helpful for preventing or treating the metabolic bone diseases. That is, the proliferation of osteoblast and the generation of a growth factor involved in bone reformation and nitric oxide are stimulated by the administration of an effective amount of the pharmaceutical composition.

The osteoblasts secret bone matrix after synthesizing thereof and is involved in bone formation by regulating the concentration of calcium and phosphorous. In a preferred embodiment of the present invention, the extract of Sophorae Fructus was confirmed to have a stimulating effect on the osteoblast proliferation. IGF-1 (insulin like growth factor-1) and TGF-β (transforming growth factor-beta) of osteoblast are included in the category of the growth factor involved in the bone reformation. IGF-1 and TGF-β stimulate osteoblast replication and promote synthesis of collagen and matrix. Especially, TGF-β inhibits the function of osteoclast but stimulates apoptosis of the osteoclast. So, bone reabsorption decreases as TGF-β increases (Spelsberg, T. C. et al., J. Mol. Endocrinol, 13, 819-828, 1999). In a preferred embodiment of the present invention, the extract of Sophorae Fructus of the invention was confirmed to have an activity to stimulate the secretion of IGF-1 and TGF-β.

According to an earlier report, nitric oxide generated in osteoblast inhibits an activity of osteoclast, resulting in the inhibition of bone reabsorption. In a preferred embodiment of the present invention, the extract of Sophorae Fructus was confirmed to have an activity to stimulate the generation of nitric oxide.

The metabolic bone diseases can be prevented or treated by administering an effective amount of a pharmaceutical composition comprising the extract of Sophorae Fructus of the present invention to a subject, since the composition works against the secretion of bone-absorptive cytokine or the osteoclast differentiation.

IL-1 beta and IL-6 are the examples of the bone-absorptive cytokines. The bone-absorptive cytokines are secreted in osteoblasts and stimulate the expression of OPG-L (osteoprotegrin ligand), an osteoclast-differentiating factor, resulting in the promotion of the osteoclast differentiation (Spelsberg, T. C., et al., Mol. Endocrinol, 13, 819-828, 1999). In a preferred embodiment of the present invention, the extract of Sophorae Fructus was confirmed to have an activity to inhibit the secretion of bone-absorptive cytokines, IL-1 beta and IL-6.

The osteoclast is attached to the surface of a bone to make an acid and a hydrolase secreted, by which bone matrix such as crystalline apatite and collagen are eliminated, resulting in the destruction of a bone. In a preferred embodiment of the present invention, the extract of Sophorae Fructus was confirmed to have an inhibitory effect on the osteoclast differentiation.

The extract of Sophorae Fructus of the present invention also has a weight gaining inhibitory effect that is caused by the lack of estrogen induced by taking out ovary from a subject. So, the present invention provides a method to inhibit weight gaining, which comprises administering an effective amount of a pharmaceutical composition comprising the extract of Sophorae Fructus of the invention to a subject.

The present invention further provides a use of the extract of Sophorae Fructus for the preparation of a medicament for preventing or the treating climacteric symptoms.

### EXAMPLES

Practical and presently preferred embodiments of the present invention are illustrated as shown in the following Examples.

However, it will be appreciated that those skilled in the art, in consideration of this disclosure, may make modifications and improvements within the spirit and scope of the present invention.

### Example 1

Preiaration of an Extract of Sophorae Fructus

20 kg of Sophorae Fructus (Jesung Pharmaceutical Co., Kyungdong Market, Korea) was pulverized into 30-mesh size by using a dry-pulverizer. Drinking water was added to the above pulverized Sophorae Fructus to lower the concentration 10 times (pulverized material:drinking water=9:1), which was heated at 100° C. for 4 hours. Then, the solution was cooled at 50° C., followed by filtering with a 100 mesh filter cloth. The solution was filtered again with a 200-mesh filter cloth to remove precipitation and to obtain filtrate. An extract of Sophorae Fructus was prepared by concentrating the supernatant using a concentrator until the volume was lowered into ⅕. The concentrate was spray-dried by a spray dryer, resulting in pulverization.

### Example 2

Preparation of an Enzyme Extract of Sophorae Fructus

The hydrothermal extract of Sophorae Frucuts prepared in the above Example 1 was filtered with a filter cloth. Amylase was added to the obtained filtrate by 0.5% (v/v), leading to an enzyme reaction at 50° C. for 16 hours. The reacting solution was concentrated using a concentrator until the volume was lowered into ⅕, resulting in the preparation of an enzyme extract of Sophorae Fructus. The concentrate was spray-dried by a spray dryer, resulting in pulverization.

### Example 3

Preparation of a Food Composition Comprisiny an Extract of Sophorae Fructus

A food composition comprising the enzyme extract of Sophorae Fructus obtained in the above Example 2 was prepared. The food composition comprising an extract of Sophorae Fructus was prepared by mixing 235 g of the enzyme extract of Sophorae Fructus obtained in the above Example 2,200 g of seaweed calcium powder (Daeduk Pharmaceutical Co., Kyunggido, Korea), 27.5 g of crystalline cellulose (Daeduk Pharmaceutical Co., Kyunggido, Korea), 5 g of hydrolyzed milk protein (Dynenatural, Seoul, Korea), 5 g of green tea ex-powder (Myung Food Co., Kyunggido, Korea), 5 g of shark cartilage extract powder (Shinil Co., Seoul, Korea), 4 g of chito-oligosaccharide (YoungDeok Chitosan Co. Ltd., Seoul, Korea), 5 g of vitamin C (Roche Vitamin Co., Seoul, Korea), 2.5 g of collagen peptide (Dynenatural, Seoul, Korea), 2.5 g of grape seeds extract powder (Daeduk Pharmaceutical Co., Kyunggido, Korea), 2.5 g of enerzyme-P (Sung Ji Corp., Kyunggido, Korea), 1 g of vitamin D3 powder (Roche Vitamin Co., Seoul, Korea) and 5 g of magnesium stearate (Dynenatural, Seoul, Korea) all together.

### Example 4

Investigation of Preventing or Treating Effects of an Extract of Sophorae Fructus on Osteoporosis through in vitro Experiments

Human osteoblasts were distributed to investigate the preventing or treating effect of an extract of Sophorae Fructus on osteoporosis. Bone marrow cells were obtained from a white rat and osteoclasts and osteoblasts were differentiated from those bone marrow cells.

Besides, other activities of the extract of Sophorae Fructus of the present invention, such as an effect on the human osteoblast proliferation, an activity to inhibit the secretion of IL-1 beta and IL-6, bone-absorptive cytokines, an activity to stimulate the secretion of IGF-1 and TGF-beta, growth factors involved in bone reformation, an effect on the generation of nitric oxide in osteoblast and an activity to inhibit the osteoclast differentiation, were also investigated. The comparison of all test groups was done by ANOVA test, and the comparison between specific test groups was done by student T-test. After establishing statistics, p value under 0.05 (P<0.05) was regarded as statistically significant.

<4-1> Cultivation of Human Osteoblasts

MG-63 human osteoblast-like cells were distributed from Korean Cell Line Bank of Seoul National University College of Medicine, Seoul, Korea, which were sub-cultured for fuirther use. The frozen MG-63 human osteoblast-like cells were melted in a 37° C. water bath for 1 minute, followed by centrifugation with 1300 rpm for 5 minutes to eliminate supernatant. The obtained pellet was re-suspended in DMEM supplemented with 10% FBS, which was, then, distributed in a 25 cm³ culture flask for further culture. The duration of culture was 2 weeks for cell stability. After confirming under a microscope that a monolayer was stably formed, the cells were used for the experiments.

<4-2> Cultivation of Osteoclasts and Osteoblasts

12-week old SD rats (Hallym Research Institute of Experimental animals, Kyunggido, Korea) were anesthetized by an over-dose of ether. Two femurs per each rats were taken out and washed several times with washing medium (15% FBS α-MEM). The final washing, though, was done with a osteoclast medium (15% FBS α-MEM containing 0.28 mM L-ascorbic acid-2-phosphate). Epiphyses of the femur were eliminated, and then, 10 ml of bone marrow cells was obtained using a 25-gauge needle.

The obtained bone marrow cells were supplemented with 10 ml of osteoclast medium, which was distributed into a 75 cm³ culture flask for further culture at 37° C. with 5% CO₂ and 100% humidity for 24 hours. After the culture, the medium was replaced with a fresh medium. The medium was replaced twice during 10 days culture and which was flurther cultured for further use.

Following the same procedure as the case of osteoclast culture, the obtained 10 ml of bone marrow cells was filtered with a 100 μm cell strainer, which was centrifuged to remove supernatant. The pellet was resuspended in a primary culture medium (15% FBS α-MEM containing 0.28 mM L-ascorbic acid-2-phosphate and 10 nM dexamethasone) by 5 ml per femur. The suspended bone marrow cells were distributed into a 75 cm³ culture flask, which was supplemented with the primary culture medium to make the volume 20 ml per femur. Cultivation was carried out at 37° C. with 5% CO₂ and 100% humidity. The medium was replaced twice on the second day and on the forth day of the culture. Trypsin was added on the sixth day and the medium was replaced with a osteoblast culture medium (15% FBS α-MEM containing 0.28 mM L-ascorbic acid-2-phosphate and 10 nM dexamethasone) for further culture.

Culture states of the osteoblasts and osteoclasts were checked by a microscope and their cell viabilities were also investigated by trypan blue dye exclusion method.

<4-3> Osteoblast Proliferation Induced by the Extract of Sophorae Fructus

In order to investigate the osteoblast proliferation after the treatment of an extract of Sophorae Fructus, MG-63 human osteoblast-like cells of the above Example <4-1> were treated with the Sophorae Fructus extract powder of Example 1 (R-G), the Sophorae Fructus enzyme extract powder of Example 2 (R-A), and the food composition comprising an extract of Sophorae Fructus of Example 3 (R-P), which were cultured for 3 days. The effects of the above extracts on cell proliferation were measured by MTT method. As comparing groups, the MG-63 osteoblast-like cells were treated with soybean ex-powder (Shin Dong Bang Corp.), 17-beta estradiol (sigma) and lipopolysaccharide (Sigma), used as a treatment agent for osteoporosis, and their effects on cell proliferation were also compared. A control group was treated with only a cell culture medium instead of the above samples. Each sample was diluted by a cell culture medium to adjust the treatment concentration to 10⁻⁴˜10⁻¹²%. LPS was added by 10 μg/ml.

MTT analysis, which is in proportion to the activity of mitochondria, was performed as follows. 1×10⁴ cells/ml of MG-63 osteoblast-like cells were distributed into a 96 well flat-bottomed tissue culture plate by 100 μl/well. Each sample was given to the plate by 10⁻⁴, 10⁻⁶, 10⁻⁸, 10⁻¹⁰ and 10⁻¹²%, followed by a reaction for 72 hours. As the reaction was completed, 10 μl of MTT (3-[4,5-dimethylthiazol-2-yl]-2,5-diphenyltetrazolium bormide) stock solution was added, which was flurther cultured at 37° C. for 4 hours. After the culture was finished, 100 μl of isopropanol/HCl was added to each well. After complete mixing, color changes were checked and OD₅₇₀ was measured using an ELISA plate reader within an hour.

As a result, similar proliferation effects were detected in the range of treating concentration in all the cases, respectively treated with the Sophorae Fructus extract powder of Example 1 (R-G), with the Sophorae Fructus enzyme extract powder of Example 2 (R-A), with the food composition comprising an extract of Sophorae Fructus of Example 3 (R-P), and with the soybean ex-powder (S-S). The confirmed effect above was higher than that of a control but lower than that of an estradiol-treating group (E group). In the case of estradiol treating group, cell proliferation depended on its concentration, and the proliferation decreased when estradiol was treated by the low concentration (10⁻¹⁰% and 10⁻¹²%). On the contrary, the proliferation in each R-G group, R-A group, R-S group and S-S group was higher with the low concentration (10⁻¹⁰%) than with the high concentration. Therefore, it was confirmed that the extract of Sophorae Fructus of the present invention promoted the osteoblast proliferation, regardless of concentration (FIG. 1).

In conclusion, the group treated with the extract of Sophorae Fructus of the present invention showed lower osteoblast proliferation effect than the group treated with estradiol, but was confirmed to stimulate the osteoblast proliferation even with the low concentration (10⁻⁶˜10⁻¹²%).

<4-4> Effect of the extract of Sophorae Fructus on the Secretion of IL-1 Beta and IL-6 in Osteoblasts

We investigated whether the extract of Sophorae Fructus of the present invention could inhibit the generation of IL-1 (interleukin-1) beta and IL-6 (interleukin-6) that were generally secreted in osteoblasts. IL-1 beta and IL-6, bone-absorptive cytokines, are secreted in osteoblasts and stimulate the expression of OPG-L (osteoprotegrin ligand), an osteoclast differentiating factor, resulting in the promotion of the osteoclast differentiation (Spelsberg, T. C., et al., Mol. Endocrinol, 13, 819-828, 1999). Thus, in case where the extract of Sophorae Fructus can inhibit the generation of IL-1 beta and IL-6, bone-absorptive cytokines, the expression of OPG-L will be inhibited, resulting in the inhibition of the osteoclast differentiation.

In order to confirm whether the extract of Sophorae Fructus of the present invention could inhibit the generation of IL-1 beta and IL-6 secreted in osteoblasts, the extract of Sophorae Fructus of Example 1 (R-G group), the enzyme extract of Sophorae Fructus of Example 2 (R-A group) and the food composition comprising the extract of Sophorae Fructus of Example 3 (R-P group) were diluted by a cell culture medium to be treated to MG-63 human osteoblast-like cells of the Example <4-1> by the concentration of 10⁻⁴˜10⁻¹⁰%. 72 hours later, the expressions of IL-1 beta and IL-6 were measured by ELISA and RT-PCR. The comparing groups were treated with soybean ex-powder (S-S group) and estradiol by the same procedure as the above, and a control group was just added with a cell culture medium.

ELISA was performed by using ELISA kit (Titerzyme ELISA kit, Assay designs) according to the provided protocol to investigate the expressions of IL-1 beta and IL-6. Then, OD₄₅₀ was measured. Each concentration was calculated by a standard curve.

RT-PCR was performed to investigate the expression of mRNAs of IL-1 beta and IL-6. Precisely, a total RNA was extracted by TRIZOL method from MG-63 human osteoblast-like cells treated with each sample by the concentration of 10⁻⁸%. Each corresponding DNA was synthesized by reverse transcription with 2 μl of the total RNA. Particularly, 12.85 μl of DEPC distilled water was mixed with 5 μl of the total RNA and 1 μl of each 10 pM primer, leading to denaturation at 72° C. for 10 minutes. Then, 0.15 μl of reverse transcriptase (5U) was added thereto, inducing a reaction at 42° C. for 10 minutes to synthesize corresponding DNAs.

Polymerase chain reaction (PCR) was performed by using the above corresponding DNAs as templates. One-stop RT-PCR premix (Accupower, Bioneer) was used to secure reproducibility and consistency of the experiment. PCR was performed using a PCR system (Dual-bay DyadTM thermal cycler system, MJ Research) with the reacting solution with 35 cycles (1 cycle: 5 minutes at 95° C., 30 seconds at 95° C., 60 seconds at 60° C., 60 seconds at 72° C.). GAPDH was used as a standard control. Amplified PCR products were quantified by using a gel documentation system, and the expression levels were represented by the comparative rates (%) to that of a control. Each primer used for the RT-PCR was presented below.

| | |
|---|---|
| Sense primer of IL-1 beta | (SEQ. ID. No 1) |

5′-AGG CAC AAC AGG CTG CTC TG-3′

| | |
|---|---|
| Antisense primer of IL-1 beta | (SEQ. ID. No 2) |

5′-TGG ACC AGA CAT CAC CAA GC-3′

| | |
|---|---|
| Sense primer of IL-6 | (SEQ. ID. No 3) |

5′-AGC GCC TTC GGT CCA GTT GC-3′

| | |
|---|---|
| Antisense primer of IL-6 | (SEQ. ID. No 4) |

5′-ACT CAT CTG CAC AGC TCT GG-3′

From the result of ELISA, it was confirmed that the secretions of IL-1 beta and IL-6 were inhibited in all of the groups except a control group; a group treated with the extract of Sophorae Fructus of Example 1 (R-G group), a group treated with the enzyme extract of Sophorae Fructus of Example 2 (R-A group), and a group treated with the food composition comprising an extract of Sophorae Fructus of Example 3 (R-P group).

When each sample was treated by the maximum concentration of 10⁻⁴% equally, the best inhibiting effect on the secretion of IL-1 beta was detected in R-P group, and the inhibiting effects of R-G group and R-A group were next but better than those of groups treated with soybean ex-powder (S-S group) and treated with estradiol. When the sample was treated by the minimum concentration of 10⁻¹⁰%, a group treated with the extract of Sophorae Fructus of the present invention showed an inhibitory effect on the secretion of IL-1 beta, and further, even when the sample was given less than comparing groups (S-S group and estradiol treated group), the group treated with the extract of Sophorae Fructus of the present invention still showed an inhibitory effect on the secretion of IL-1 beta. Especially, the secretion of IL-1 beta (60 pg/ml) in R-P group was all equal in every case given different concentrations (FIG. 2A).

Both when each sample was treated by the maximum concentration of 10⁻⁴% and when treated by the minimum concentration of 10⁻¹⁰%, the best inhibitory effect on the secretion of IL-6 was detected in R-G group, which was treated with the extract of Sophorae Fructus of Example 1. Especially, in the case of R-P group, the secretion levels of IL-6 (110 pg/ml) in the cases given different concentrations were all equal. Thus, the extract of Sophorae Fructus of the present invention was confirmed to have IL-6 inhibiting activity even with the lower concentration than those of S-S group and estradiol treating group (FIG. 2B).

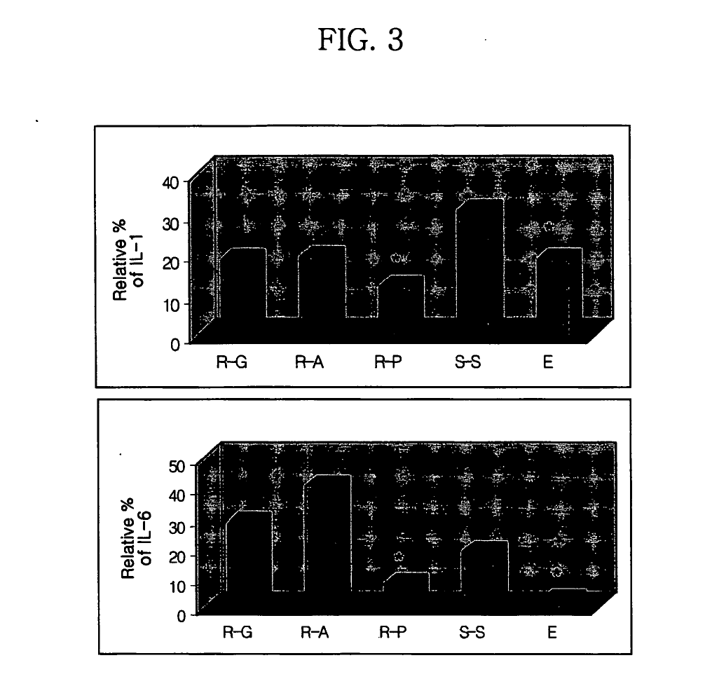

The result of RT-PCR has a similar pattern to that of ELISA. The best inhibitory effect on the expression of IL-1 beta was detected in R-P group, which was treated with the food composition comprising the extract of Sophorae Fructus. And, a group treated with the extract of Sophorae Fructus of Example 1 (R-G group) and a group treated with the enzyme extract of Sophorae Fructus of Example 2 (R-A group) showed similar effects to estradiol treating group (E group). The lowest inhibitory effect on the expression of IL-1 beta was observed in a group treated with soybean ex-powder (S-S group). The inhibitory effect on the expression of IL-6 observed in R-P group was also excellent, which was actually better than that seen in S-S group (FIG. 3).

Therefore, the extract of Sophorae Fructus of the present invention was confirmed to inhibit the osteoclast differentiation by suppressing the secretion of IL-1 beta and IL-6, and such inhibitory activity was detected even with a low concentration, unlike other conventional food compositions or medicines for the treatment of osteoporosis.

<4-5> Effect of the Extract of Sophorae Fructus on the Secretion of IGF-1 and TGF-beta in Osteoblasts

We investigated whether the extract of Sophorae Fructus of the present invention could stimulate the secretion of IGF-1 (insulin like growth factor-1) and TGF-beta (transforming growth factor-beta) in osteoblasts. IGF-1 and TGF-beta, growth factors involved in bone reformation, are known to stimulate the osteoblast replication and to enhance the synthesis of collagen and a matrix. Particularly, TGF-beta inhibits the functions of osteoclast but stimulates apoptosis of osteoclast. So, bone reabsorption decreases as TGF-beta increases (Spelsberg, T. C. et al., J. Mol. Endocrinol, 13, 819-828, 1999).

In order to investigate whether the extract of Sophorae Fructus of the present invention could stimulate the secretion of IGF-1 and TGF-beta in osteoblasts, each sample was treated to MG-63 human osteoblast-like cells by the same method as used in Example <4-4>, and then, ELISA and RT-PCR were performed to measure the secretion and the expression of IGF-1 and TGF-beta. The expression levels of IGF-1 and TGF-beta were represented by a comparative rate (%) to that of a control.

ELISA was performed by using ELISA kit (Quantikine, R&D system) according to the provided protocol to investigate the secretion of IGF-1 and TGF-beta. Then, optical density was measured by the same method as used in the Example <4-4>. RT-PCR was also performed by the same method as used in the above Example <4-4>. Each primer used for the RT-PCR was presented below.

| | |
|---|---|
| Sense primer of TGF-beta | (SEQ. ID. No 5) |

5′-CGC CCT GTT CGC TCT GGG TAT-3′

| | |
|---|---|
| Antisense primer of TGF-beta | (SEQ. ID. No 6) |

5′-AGG AGG TCC GCA TGC TCA CAG-3′

| | |
|---|---|
| Sense primer of IGF-1 | (SEQ. ID. No 7) |

5′-ATG CTC TTC AGT TCG TGT GT-3′

| | |
|---|---|
| Antisense primer of IGF-1 | (SEQ. ID. No 8) |

5′-AGC TGA CTT GGC AGG CTT GT-3′

From the result of ELISA, it was confirmed that the concentration of IGF-1 was high in every test groups than that of a control group. When each sample was treated by the high concentration of 10⁻⁴%, the highest IFG-1 concentration was detected in an estradiol treating group (E group), and when each sample was treated by 10⁻⁶% concentration, the highest IGF-1 concentration was detected in a group treated with the extract of Sophorae Fructus of Example 1 (R-G group). Even when each sample was treated by the low concentration of 10⁻¹²%, IGF-1 concentration was still high in the group treated with the extract of Sophorae Fructus of Example 1 (R-G group), in a group treated with the enzyme extract of Sophorae Fructus of Example 2 (R-A group) and in a group treated with the food composition comprising the extract of Sophorae Fructus of Example 3 (R-P group), which were all higher than that in an estradiol treating group (FIG. 4A).

As for TGF-beta concentration, when each sample was treated by the concentration ranging from 10⁻⁴ to 10⁻¹⁰%, the estradiol-treating group showed the evenly higher TGF-beta concentration, suggesting that the estradiol had a TGF-beta promoting activity. But, when estradiol was treated by the low concentration of 10⁻¹²%, TGF-beta concentration decreased to the similar level to that in a control group. However, even when R-G group which was treated with the extract of Sophorae Fructus of the present invention and R-A group which was treated with the enzyme extract of Sophorae Fructus were treated by the minimum concentration of 10⁻¹²%, activities of promoting the secretion of TGF-beta were still high (FIG. 4B).

In conclusion, when estradiol was treated, its pharmaceutical effect on the secretion of IGF-1 and TGF-beta decreased dose-dependently. On the other hand, when the extract of Sophorae Fructus of the present invention was treated (R-G, R-A, and R-P group), its effect was still the same even with the low concentration. Especially in the R-P group treated with the food composition comprising the extract of Sophorae Fructus of Example 3, the secretions of IGF-1 and TGF-beta were evenly promoted in all the groups treated by different concentrations respectively.

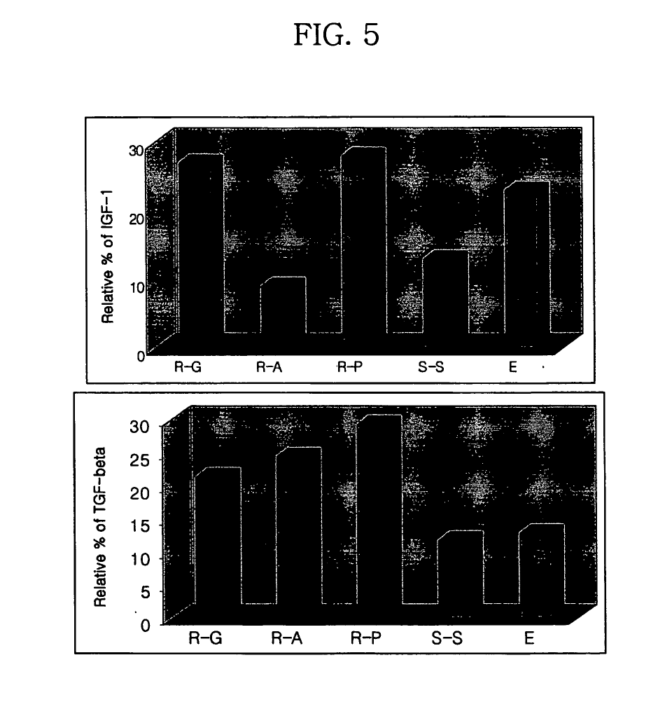

RT-PCR was performed with RNA extracted from cells treated with each sample by 10⁻⁸% concentration. As a result, the expression of IGF-1 was higher in R-G group treated with the extract of Sophorae Fructus of the Example 1 and in R-P group treated with the food composition comprising the extract of Sophorae Fructus of the Example 3 than in E group treated with estradiol. The expression of TGF-beta was also higher in R-P group, in R-A group and in R-G group than in E group treated with estradiol (FIG. 5).

Therefore, it was confirmed that the extract of Sophorae Fructus of the present invention promoted the expression of IGF-1 and TGF-beta dose-independently. In other word, the extract of Sophorae Fructus of the present invention could promote the expression of IGF-1 and TGF-beta, and inhibit the fimction of osteoclast even with a low concentration.

<4-6> Effect of the Extract of Sophorae Fructus on the Generation of Nitric Oxide in Osteoblasts

We investigated the effect of the extract of Sophorae Fructus of the present invention on the generation of nitric oxide (NO). The nitric oxide has been known to play an important role in regulation of bone loss, in particular, bone resorption into blood. In other word, nitric oxide, secreted in osteoblasts, has been reported to inhibit the resorption of bone by suppressing the osteoclast activity (Ralston S. H. et al., Endocrinology, 135, 330˜336, 1994; Vant Hof R. J. et al., Immunol., 103, 255˜261, 2001).

Thus, the generation of nitric oxide and the expression of ecNOS (endothelial nitric oxide synthase), a nitric oxide generating enzyme, were examined in order to investigate the effect of the extract of Sophorae Fructus on the generation of nitric oxide. The expression level of ecNOS was represented by comparative ratio (%) to a control.

Following the same procedure as used in the above Example <4-4>, each sample was treated to MG-63 human osteoblast-like cells by 10⁻⁴˜10⁻¹²% concentration. The amount of generated nitric oxide was measured by ELISA. A total RNA was extracted by the same method as used in the above Example <4-4> from the cells treated with the samples by 10⁻⁸%. The amount of generated ecNOS was measured through RT-PCR. Primers used for RT-PCR were represented in the below.

| | |
|---|---|
| Sense primer of ecNOS | (SEQ. ID. No 9) |

5′-AAG CCG CAT ACG CAC CCA GAG-3′

| | |
|---|---|
| Antisense primer of ecNOS | (SEQ. ID. No 10) |

5′-TGG GGT ACC GCT GCT GGG AGG-3′

It was proved by ELISA that when each sample was treated by the high concentration of 10⁻⁴%, the generation of nitric oxide was highest in S-S group treated with soybean ex-powder. But, when each sample was treated by the concentration ranging from 10⁻⁶ to 10⁻¹⁰%, the generation of nitric oxide was significantly higher in R-P group treated with the food composition comprising the extract of Sophorae Fructus of Example 3 than in S-S group. And when each sample was treated by the low concentration of 10⁻¹⁰˜10⁻¹²%, the generations of nitric oxide in groups treated with the extract of Sophorae Fructus (R-G, R-A) and a group treated with the food composition comprising the extract of Sophorae Fructus of Example 3 (R-P group) were all greater than in an estradiol treating group(E group) (FIG. 6).

Also, form the result of RT-PCR, it was confirmed that the expression of ecNOS was highest in R-P group.

Thus, the extract of Sophorae Fructus of the present invention and the food composition comprising the same were proved to stimulate the generation of nitric oxide and the expression of ecNOS, which was not changed with the low concentration. In addition, such activities of the extract were confirmed to be similar or superior to that of estradiol.

<4-7> Inhibitory Effect of the Extract of Sophorae Fructus on the Osteoclast Differentiation

We investigate the inhibitory effect of the extract of Sophorae Fructus of the present invention on the osteoclast differentiation. Osteoclasts and osteoblasts, separated and pre-cultured in the above Example <4-2>, were co-cultured for that purpose. Particularly, osteoclasts were distributed in a 24 well plate (multiwell™ 24 well, Becton Dickinson) by 1.5×10⁵/well, into which osteoblasts were distributed by 1×10³cells/well. The wells were treated with M-CSF, a differentiating factor, by 50 ng/ml along with each sample, which were, then, cultured for 5 days. Each sample was prepared by diluting the extract of Sophorae Fructus of the Example 1 (R-G group), the enzyme extract of Sophorae Fructus of the Example 2 (R-A group) and the food composition containing the extract of Sophorae Fructus of the Example 3 (R-P group) degrees by degrees to make 10⁻⁴, 10⁻⁶, 10⁻⁸, 10⁻¹⁰, and 10⁻¹²% concentration each. After finishing the culture, the osteoclast differentiation was investigated by TRAP (tartrate-resistant acid phosphatase) staining method, which was counting nuclei positive to TRAP by using acid phosphatase kit (Sigma) through an optical microscope.

As a result, the inhibitory effect of the extract of Sophorae Fructus of the present invention on the osteoclast differentiation was inferior to that of estradiol. However, when each sample was treated by the high concentration of 10⁻⁴ and 10⁻⁶%, the inhibitory effect on the osteoclast differentiation was excellent in a group treated with the extract of Sophorae Fructus of Example 1 (R-G group) and in a group treated with the food composition comprising the extract of Sophorae Fructus of Example 3 (R-P group), comparing to a group treated with soybean ex-powder (S-S group). Even when each sample was treated with the low concentration of 10⁻⁸%˜10⁻¹²%, the effect was still the same in groups treated with the extract of Sophorae Fructus (R-G, R-A and R-P group), which was higher than that in a group treated with soybean ex-powder (S-S group). The inhibitory effect on the osteoclast differentiation in R-P group was all equal with different concentrations (FIG. 8 and FIG. 9).

Therefore, the extract of Sophorae Fructus of the present invention and the food composition comprising the same were confirmed to have an inhibitory effect on the osteoclast differentiation.

### Example 5

Investigation of the Effect of the Extract of Sophorae Fructus on the Prevention and the Treatment of Osteoporosis through in vivo Experiments

The effect of the extract of Sophorae Fructus of the present invention on the prevention and the treatment of osteoporosis was investigated by animal tests. Ovary was ectomized from white rats to cause osteoporosis. While the extract of Sophorae Fructus was being administered to the white rat, weight changes, growth rate, changes of Dpd (Deoxypyridinoline) and Ca (Calcium) concentration, indexes of bone replacement rate in serum, were measured. Besides, changes of the size of trabecular bone of the tibia and the lumbar of the rats were also investigated while the extract of Sophorae Fructus was administered continuously. The comparison among all test groups was done by ANOVA test, and the comparison among specific groups was done by student-T test. After establishing statistics, p value under 0.05 was regarded as statistically significant.

<5-1> Removal of Ovary from an Experimental Animal

As the experimental animals, white female SD (Sprague-Dawley) rats (230˜250 g) were purchased from Hallym Research Institute of Experimental animals (Kyunggido, Korea). The rats were raised at 23±1° C., under 40˜60% humidity and 12-hour light and shade cycle. Feed (solid feed, Hallym Research Institute of Experimental animals, Kyunggido, Korea) and water were given freely, but just water was supplied on the day before drawing blood.

For removal of ovary, a 12-week old white rat was anesthetized by ether. The rat got a shave on its back by a razor. Oophorectomy was performed after sterilizing the operating area with 70% ethanol. Particularly, skin was cut 2˜3 cm along the spinal column of the abdominal lower-flank region of one side, and then, muscle and peritoneum were incised 1.5 cm to exposure ovary. After ligating oviduct by a silk, the ovary was cut off and peritoneum, muscle and skin were sutured by a silk. The other side ovary was ectomized by the same procedure. Sham operation was performed for a control group, which opened peritoneum but sutured without taking ovary out. A period of recovery was 1 week.

<5-2> Administration of Each Sample

Test animals were classified into three groups; a normal group (non-ovari-ectomized group), a control group 1 (sham-operated group) and an ovari-ectomized group. The ovari-ectomized group was sub-divided into a control group 2, which was not administered with a sample, a 17-beta estradiol treating group (E group), an extract of Sophorae Fructus of Example 1 administering group (R-G group), an enzyme extract of Sophorae Fructus of Example 2 administering group (R-A group), a food composition comprising the extract of Sophorae Fructus of Example 3 administering group (R-P group) and a soybean ex-powder administering group (S-S group) by 10 animals each (Table 1). Administration dosages of each sample were presented below, and the duration of administration was 9 weeks, which began a week after operation to a 13-week old white rat and finished when the rat became 22 weeks old.

TABLE 1
Administration dosage and method

| Group | Dose and method |
|---|---|
| Normal group (non-ovari- | Not administered |
| ectomized) | |
| Control group 1 (sham-operated) | Drinking water 1 ml/day, oral |
| | administration |
| Control group 2 (ovari-ectomized) | Drinking water 1 ml/day, oral |
| | administration |
| E group | 1 g/kg/day, intraperitoneal injection |
| R-G group | 0.556 g/kg/day, oral administration |
| R-A group | 0.556 g/kg/day, oral administration |
| R-P group | 0.556 g/kg/day, oral administration |
| S-S group | 0.556 g/kg/day, oral administration |

<5-3> Measurement of Weight Changes and Growth Rate

Weights of each group of the above Example <5-2> were measured with an electronic scale, from which weight gaining rate per day was calculated by the below formula.
Weight gaining rate per day=(final weight−beginning weight)/experiment day×100

As a result, there was no statistically significant difference among the groups before oophorectomy. But, rapid weight gaining was observed in a control group 2 that was given only drinking water after ovary was ectomized. On the other hand, weight gaining was slow in a normal group, which kept ovary not to be ectomized, in a control group 1 (sham-operated group) and in a group administered with an extract of Sophorae Fructus or estradiol (FIG. 10 and Table 2). In conclusion, when ovary was ectomized from a white rat, estrogen was no more secreted, leading to rapid weight gaining by the increase of fat cells. However, when the extract of Sophorae Fructus and estradiol were administered after the removal of ovary, such samples took the place of estrogen to inhibit the increase of fat cells, resulting in slow weight gaining.

TABLE 2
Weight gaining rate per day

| | Weight (g) | | |
|---|---|---|---|
| | Beginning | Final | Weight |
| | weight (12 | weight (22 weeks | gaining rate |
| Group | weeks old) | old) | (g/day) |
| Normal group (non- | 231.4 ± 12.303¹⁾ | 294.2 ± 21.869 | 0.9968 ± 0.1*²⁾ |
| ovari-ectomized) | | | |
| Control group 1 | 242.1 ± 17.520 | 280.8 ± 14.336 | 0.6142 ± 0.2 |
| (sham-operated) | | | |
| Control group 2 (ovari- | 236 ± 8.5147 | 335.6 ± 34.112 | 1.5809 ± 0.2 |
| ectomized) | | | |
| Estradiol treating | 238.8 ± 16.742 | 302.4 ± 29.885 | 1.0095 ± 0.1* |
| group | | | |
| R-G group | 241.2 ± 7.159 | 306.9 ± 11.662 | 1.0428 ± 0.1* |
| R-A group | 244.3 ± 8.355 | 319.6 ± 23.697 | 1.1952 ± 0.1 |
| R-P group | 243.2 ± 12.903 | 307.2 ± 25.772 | 1.0158 ± 0.1* |
| S-S group | 245.6 ± 9.341 | 322.1 ± 19.548 | 1.2142 ± 0.2 |

¹⁾Mean ± SD(standard deviation).
*²⁾Statistically significant when p < 0.05.

<5-4> Changes of Dpd Concentration in Blood Plasma after the Administration of the Extract of Sophorae Fructus

We investigated changes of Dpd (deoxypyridinoline) concentration in blood plasma after the administration of the extract of Sophorae Fructus. The Dpd plays an important role in stabilization of type 1 collagen chain by forming cross-link in a matrix of bone (Seyedin S M. et al., Curr. Opin. CellBiol. 2, 914-919, 1990; Delmas P D. Biochemical markers for the assessment of bone turnover. In Riggs B L, Melton L J, Osteoporosis; etiology, diagnosis, and management Philadelphia; Lippincott-Raven Publishers, 319-333, 1995). When a matrix of bone is decomposed by osteoclasts, Dpd is excreted through urine (Eastell R. et al., J. Bone Miner. Res. 12, 59-65, 1997). Thus, inhibition of the increase of Dpd positively affects the prevention or the treatment of metabolic bone diseases (Riggs B L., West. J. Med. 154, 63-77, 1991; Hesley R P. et al., Osteoporosis int. 8, 159-164, 1998).

In order to investigate whether the administration of the extract of Sophorae Fructus could inhibit the increase of Dpd in blood plasma, blood plasma was obtained from the experimental animals of Example <5-2>. 1.7˜1.8 ml of blood was taken from orbital vein of a white rat after anesthetizing the animal by ether, every other week starting before oophorectomy(12-week old, on 0^{th} week of the test). Each sample was administered for 9 weeks. Before sacrificing the rat (22-week old, on the 10^{th} week of the test), blood was taken again from ventral vein, which was centrifuged right away to obtain blood plasma.

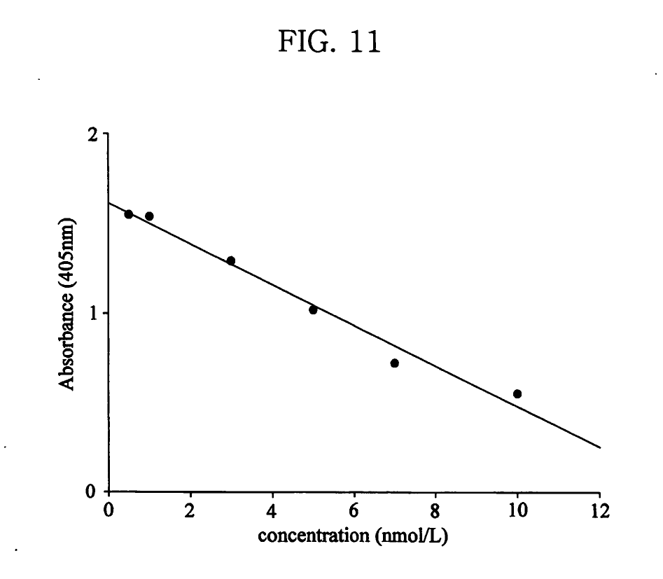

Dpd concentration in blood plasma obtained above was measured by competitive enzyme immunoassay using a Dpd concentration measuring kit (Pyrilinks-D, Quidel Corporation, USA) in which an anti-Dpd antibody was included. Precisely, microtiter strip wells were coated with anti-Dpd antibody, inducing a competitive reaction between Dpd in blood plasma and Dpd-alkaline phosphatease conjugate. As a substrate, p-nitrophenyl phosphate (PNPP) was added thereto for further reaction. After reaction was finished, OD₄₅₀ was measured. Then, Dpd concentration was calculated by using a calibration curve made to explain the relation between Dpd content and optical density (FIG. 11).

As a result, Dpd concentration in blood plasma was hardly changed for 10 weeks in the normal group (having ovary) and a control group 1 (sham operated group). As for a control group 2 administered with drinking water after the removal of ovary, Dpd concentration in blood plasma increased fast and continuously, which was about 60% increase, comparing to the normal group. Such result supported the belief that osteoporosis became serious because of the decrease of secretion of estrogen, which was caused by the removal of ovary. Dpd concentrations were increased in a group administered with estradiol (E group), in a group administered with the extract of Sophorae Fructus of Example 1 (R-G group), in a group administered with the enzyme extract of Sophorae Fructus of Example 2 (R-A group), in a group administered with the food composition comprising the extract of Sophorae Fructus of Example 3 (R-P group) and in a group administered with soybean ex-powder (S-S group) only during the first week of the test when each sample was not administered yet after oophorectomy. However, Dpd concentrations in those groups were decreased after 9 weeks (on the 10th week) from starting administering each sample. In particular, rapid drop of Dpd concentration was observed in R-G group and a group treated with estradiol (E group) (FIG. 12).

Therefore, it was confirmed that the extract of Sophorae Fructus of the present invention had an inhibitory activity to increase Dpd concentration.

<5-5> Confirmation of the Inhibitory Effect of the Extract of Sophorae Fructus on the Increase of Dpd Concentration

Based on the result of the above Example <5-4>, changes of Dpd concentration after administering each sample for 9 weeks were quantified by the below formula. A negative ΔDpd value means the decrease of Dpd concentration, and a positive ΔDpd value means the increase of Dpd concentration.
ΔDpd=total of ΔDpd of Individual animals/n

In the above formula, ΔDpd of each animal means the difference in Dpd before the administration of each sample and after 9-week administration of the sample, and “n” means the number of experimental animals.

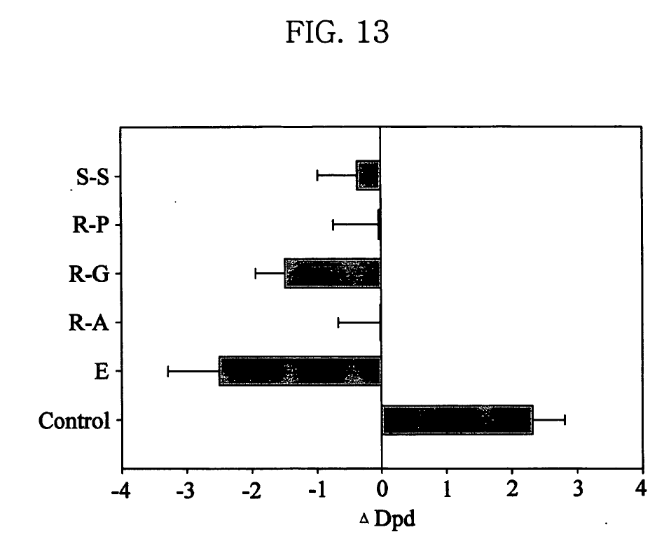

From the result of calculating ΔDpd value, it was confirmed that ospeoporosis in a control group 2, which was administered with just drinking water, was much progressed, supported by the highly positive ΔDpd value. On the other hand, ΔDpd values of R-G group and R-A group, which were administered with the extract of Sophorae Fructus, R-P group that was administered with the food composition comprising the extract of Sophorae Fructus, E group that was administered with estradiol and S-S group that was administered with soybean ex-powder were all negative, suggesting that the samples had an Dpd inhibitory effect. Dpd was best inhibited in E group administered with estradiol. Regarding the amount of decreased Dpd of E group as 100%, the decrease rate of Dpd in R-G group, in S-S group, in R-P group and in R-A group was 60%, 14.5%, 1.2% and 0.7% respectively (FIG. 13). Therefore, it was sure that R-G group showed greater Dpd decrease activity than S-S group.

In order to confim the effects of the extract of Sophorae Fructus of the present invention, Dpd concentrations in groups administered with the extract of Sophorae Fructus of the present invention were calculated by comparing with Dpd concentration in a control group 2 (ovari-ectomized) in which osteoporosis was progressed much. The calculation was carried out by the below formula.
Effect of the extract of Sophorae Fructus=(ΔDpd_{control group 2}−ΔDpd experimental group)/ΔDpd_{control group 2}

In the above formula, ΔDpd_{control group} means the difference between Dpd concentration before the administration of sample and final Dpd in a control group 2.

ΔDpd_{experimental group} means the difference between Dpd concentration before the administration of each sample and final Dpd concentration in an experimental group.

When the calculated value was over 1, the extract of Sophorae Fructus of the present invention was regarded as having Dpd inhibitory effect. The bigger the value was, the greater the effect of the extract was. ANOVA test was performed to determine statistical significance of the value at p=0.05.

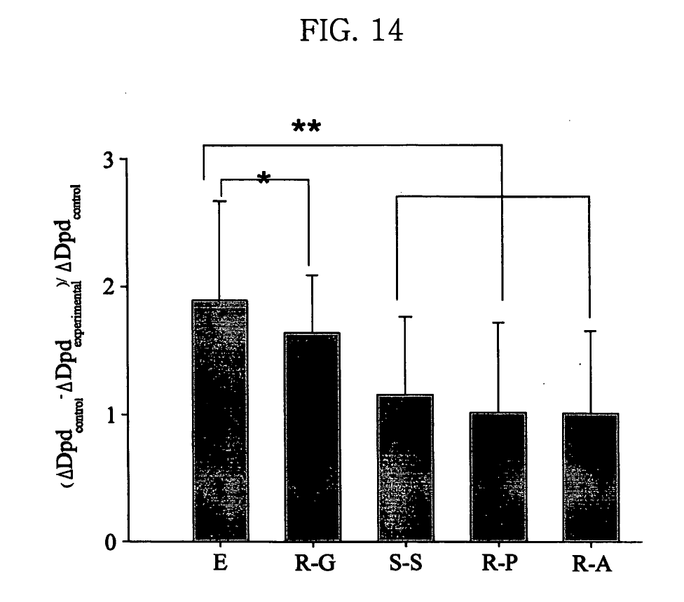

As a result, the effect of the extract in R-G group was similar to that in an estradiol treating group and the effects in other groups were all inferior to that in an estradiol treating group (FIG. 14).

<5-6> Changes of Calcium Concentration in Blood Plasma after the Administration of the Extract of Sophorae Fructus

In general, increase of calcium concentration reflects bone formation, making it an index for bone formation. Thus, changes of calcium concentration in blood plasma after the administration of an extract of Sophorae Fructus were investigated by OCPC method (J. P. Riley, Analytica Chimica Acta, 21, 317-323, 1959). Blood plasmas were obtained from experimental animals following the same method as used in Example <5-4>. Calcium in blood plasma becomes magenta color when it is linked to OCPC under the condition of alkali. Therefore, measuring optical density of the magenta color leads to the quantification of calcium in a reagent. In the embodiment of the present invention, 0.88 mol/l monoethanolamine (pH 11.0) was used as a buffer solution, and 0.1 mmol/l of OCPC and 11 mmol/l of 8-hycroxy quinoline-5-sulfonic acid were used as coloring reagents.

As a result, calcium concentration in a normal group (non-ovari-ectomized) and in a control group 1 (sham-operated group) increased gradually, which seemed to be resulted from the growth of the experimental animal. As for a control group 2 administered with only drinking water after the removal of ovary, calcium concentration decreased gradually. In the case of other groups; R-G group administered with the extract of Sophorae Fructus of Example 1, R-A group administered with the enzyme extract of Sophorae Fructus of Example 2, R-P group administered with the food composition comprising the extract of Sophorae Fructus of Example 3, E group administered with estradiol and S-S administered with soybean ex-powder, calcium concentration decreased gradually until the administration of each sample after the removal of ovary (on the first week), but from then on, calcium concentration turned to increase continuously. Especially, rapid increase of calcium concentration was observed in an E group (estradiol treated group) and in a R-G group (FIG. 15 and Table 3).

TABLE 3
Changes of calcium concentration by the extract of Sophorae Fructus

| | 0 week | 1 week | 10 week |
|---|---|---|---|
| Normal group | 10.333 ± 0.1003¹⁾ | 10.01 ± 0.1391 | 11.386 ± 0.571 |
| (non-ovari-ectomized) | | | |
| Control group 1 | 10.064 ± 0.0678 | 9.322 ± 0.177 | 10.94 ± 0.853 |
| (sham-operating group) | | | |
| Control group 2 | 10.2 ± 0.1011 | 9.188 ± 0.222 | 9.65 ± 0.677 |
| (ovari-ectomized) | | | |
| Estradiol treating group | 10.217 ± 0.1211 | 8.48 ± 0.344 | 17.96 ± 1.334 |
| R-G | 9.892 ± 0.0753 | 8.656 ± 0.389 | 16.386 ± 0.743 |
| R-A | 10 ± 0.1106 | 8.12 ± 0.384 | 13.463 ± 1.210 |
| R-P | 9.967 ± 0.1003 | 8.11 ± 0.339 | 14.125 ± 0.872 |
| S-S | 10.483 ± 0.3619 | 8.6 ± 0.219 | 11.233 ± 1.1938 |

¹⁾Mean ± SD (standard deviation)

<5-7> Measurement of the Area of Trabecular Bone of the Tibia and the Lumbar in Accordance with the Administration of an Extract of Sophorae Fructus

In order to investigate the effect of the extract of Sophorae Fructus of the 15 present invention on the bone density, the area of trabecular bone of the tibia and the lumbar was measured. Bone metabolism takes place in the trabecular bone most actively, so that bone formation and bone absorption were affected fastest by outside stimulus. So, measuring the size of trabecular bone leads to the judgment of the effect of the extract on osteoporosis (Faugere M C. et al., American Physiological Society, E35-E38, 1986).

In order to measure the area of trabecular bone of the tibia and the lumbar, which might be varied from the administration of the extract of Sophorae Fructus, white rats of each testing group of Example <5-2> were sacrificed to take the tibia and the lumbar out. The obtained tibia and the lumbar were fixed in 10% formalin solution. Decalcification was performed in formic acid and targeting regions of the bone were cut by a surgical knife. After dehydration step by step from 70% to 100% alcohol and acetone, the sections were embedded by paraffin. The embedded bone tissues were cut 5 micron by a microtome. Hematoxyline eosin staining (H&E staining) was performed for observation under an optical microscope (Olympus BH-2), followed by measurement of epiphyses of the tibia and the lumbar quantitatively and morphometrologically.

For the measurement, images were obtained through 1× objective lens of an optical microscope (Olympus BH-2) using a polaroid digital camera. Out line of each trabecular bone was drawn on computer and an automatic calculating program was used. All the trabecular bones in secondary ossificating region right under the epiphyseal plate of epiphysis were measured. The size was calculated automatically on computer, which was then analyzed by an image analysis system (Optimas ver 6.2, Media Cybernetics. Inc.). The mean value was obtained based on the established statistics, and the size of trabecular bone among a whole size of a targeting area was quantified by %.

As a result, as for the tibia, the area of tarbecular bone was less reduced in R-G group administered with the extract of Sophorae Fructus of Example 1, in R-A group administered with the enzyme extract of Sophorae Fructus of Example 2 and in R-P group administered with the food composition comprising an extract of Sophorae Fructus of Example 3 than in a control group 2 (ovari-ectomized) administered with drinking water, and bone density of those groups were similar or superior to group E administered with estradiol. In particular, the diminution of the area of trabecular bone in R-P group was remarkably inhibited, reflecting an excellent inhibitory effect on ospeoporosis (FIG. 16A and 16B). As for the lumbar, the area of tarbecular bone was less reduced in groups administered with the extract of Sophorae Fructus of the present invention (R-A and R-P group) than in a control groups (FIG. 17A and 17B).

The entire disclosure of Korea Patent Application No. 2003-0084329, filed on Nov. 26, 2003 including its specification, claims, drawings and summary are incorporated herein by reference in its entirety.

### INDUSTRIAL APPLICABILITY

As explained above, the extract of Sophorae Fructus of the present invention has activities of promoting the osteoblast proliferation, inhibiting the secretion of bone-absorptive cytokines, promoting the secretion of growth factors involved in bone-reformation, stimulating the generation of nitric oxide in osteoblasts and inhibiting the osteoclast differentiation. In addition, the extract lowers the concentration of a substance used as an index for bone absorption and inhibits the decrease of calcium concentration and the decrease of bone density. Therefore, the extract of Sophorae Fructus of the present invention can be effectively used for the prevention or the treatment of climacteric symptoms including osteoporosis.

1. A pharmaceutical composition for preventing or treating metabolic bone disease comprising a hot water extract of Sophorae Fructus extract as an effective ingredient.

2. The pharmaceutical composition of claim 1, wherein said hot water extract of Sophorae Fructus is prepared by the steps of:
(a) adding water to the Sophorae Fructus powder, wherein the amount of water is 3 to 20 times as much as the weight of Sophorae Fructus powder; and
(b) hydrothermal extracting the composition of step (a) for 1 to 6 hours to obtain the hot water extract of Sophorae Fructus.

3. The pharmaceutical composition of claim 1, wherein said hot water extract of Sophorae Fructus is prepared by the steps of:
(a) adding water to the Sophorae Fructus powder, wherein the amount of water is 3 to 20 times as much as the weight of Sophorae Fructus powder;
(b) hydrothermal extracting the composition of step (a) for 1 to 6 hours to obtain the hot water extract of Sophorae Fructus; and
(c) adding amylase or pectinase to the hot water extract of Sophorae Fructus of the step (b) by 0.01˜1% (v/v), and reacting for 4˜24 hours.

4. The pharmaceutical composition of claim 1, wherein the metabolic bone disease is selected from a group consisting of osteoporosis, lumbago, rheumatoid arthritis, degenerative arthritis, rickets, osteomalacia and Paget's disease of bone.

5. A food composition for preventing or improving metabolic bone disease comprising a hot water extract of Sophorae Fructus an effective ingredient.

6. The food composition of claim 5, wherein the metabolic bone disease is selected from the group consisting of osteoporosis, lumbago, rheumatoid arthritis, degenerative arthritis, rickets, osteomalacia and Paget's disease of bone.

7. A method of preventing or treating metabolic bone disease, which comprises administering a pharmaceutical composition comprising a hot water extract of Sophorae Fructus to a subject.

8. The method of claim 7, wherein the metabolic bone disease is selected from the group consisting of osteoporosis, lumbago, rheumatoid arthritis, degenerative arthritis, rickets, osteomalacia and Paget's disease of bone.

9. (canceled)

10. The method of claim 9, wherein the metabolic bone disease is prevented or treated by stimulating the osteoblast proliferation, the secretion of a growth factor involved in bone reformation, and the generation of nitric oxide in the osteoblast by the administration of the pharmaceutical composition comprising the hot water extract of Sophorae Fructus to a subject.

11. The method of claim 10, wherein the growth factor involved in bone reformation is IGF-1 or TGF-β.

12. The method of claim 7, wherein the metabolic bone disease is prevented or treated by inhibiting the secretion of bone-absorptive cytokines or the osteoclast differentiation by the administration of the pharmaceutical composition comprising the hot water extract of Sophorae Fructus to a subject.

13. The method of claim 12, wherein the bone-absorptive cytokine is IL-1 beta or IL-6.

14. (canceled)

15. Use of a hot water extract of Sophorae Fructus for the preparation of a medicament for preventing or treating metabolic bone disease.
